(19) United States
(12) Patent Application Publication
(10) Pub. No.: US 2017/0351752 A1
(43) Pub. Date: Dec. 7, 2017

(54) SYSTEMS AND METHODS FOR IDENTIFYING AND CLASSIFYING TEXT

(71) Applicant: Panoramix Solutions, Minneapolis, MN (US)

(72) Inventors: Mary Meehan, Minneapolis, MN (US); Adam Elliott, Lino Lakes, MN (US); Sudheer Prem, Apple Valley, MN (US)

(21) Appl. No.: 15/616,180

(22) Filed: Jun. 7, 2017

Related U.S. Application Data
(60) Provisional application No. 62/346,602, filed on Jun. 7, 2016.

(51) Int. Cl. G06F 17/30

(52) U.S. Cl. CPC G06F 17/30707; G06F 17/30675; G06F 17/30696

(57) ABSTRACT

Systems and methods for analyzing a plurality of data records to provide a comprehensive understanding of the data. For example, one or more public or private databases may be searched based on a user's search term(s). The results from the search may be analyzed to determine values, categories, core trends, concepts, and/or clusters present within the search results. The search results may be grouped or organized based on the values, categories, core trends, clusters, and/or concepts, and may be presented to a user via a user interface. Additionally, systems and methods of the present disclosure relate to analyzing public or private company data, and tracking or monitoring the data over time to provide real time analysis. For example, customer reviews, complaints, social media posts, or other data related to a company or product may be analyzed to determine values, categories, core trends, concepts, and/or clusters.

### CROSS-REFERENCE TO RELATED APPLICATIONS

The present application claims priority to U.S. Provisional Application No. 62/346,602, entitled Systems and Methods for Identifying and Classifying Text, and filed on Jun. 7, 2016, the content of which is hereby incorporated by reference herein in its entirety.

### FIELD OF THE INVENTION

The present disclosure relates to data analysis. Particularly, the present disclosure relates to identifying and classifying text in a database. More particularly, the present disclosure relates to the use of values, categories, core trends, concepts, and clusters for evaluating a plurality of data records to present a comprehensive view of the data.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Consumers, marketers, advertisers, content managers, other businesspersons, and internet users regularly search the internet, or other databases, for information related to a particular search topic using a search engine. Depending on the search engine used, the search engine will return the most relevant documents or links related to the search topic entered. These documents or links can take the form of various information sources such as articles, blogs, videos, tweets, status updates, social media posts, webpages, and other sources of data and information. The search engine may display relevant documents in some order, but these documents are not organized in a way that an internet user such as a consumer or marketer can readily ascertain meaning. Instead, the internet user must open or otherwise access these documents or links and read the information to uncover their meaning. The user can then repeat this process to develop a more comprehensive meaning of all of the relevant documents from the search. While this manual process of searching and reading can result in a more comprehensive understanding of the topic of interest, it is not optimal.

One way that search engines have attempted to categorize documents is by creating tag clouds or word clouds based on search results or trending topics. Tag clouds are a way to visually represent data from a document or a set of documents. Tag clouds use semantic technology to identify the most pertinent words in that document or set of documents. The most pertinent words are then displayed within the tag cloud in different font sizes based on their relevance. In this case, it tells the user which words are most relevant by putting those words that appear most frequently in larger text than the words surrounding it. Tag clouds therefore may provide information about the search topic, but they do not allow the user to derive meaning from the search. Further, tag clouds do not allow the user to define what meaning they are looking to derive from the search.

Additionally, companies routinely review and analyze customer data, product review data, social media data, company review data, and/or other data related to the company. In some cases, this company data may be reviewed or analyzed by reviewing each data entry or item individually. However, individual review may require a relatively substantial amount of time. Moreover, in order to obtain a comprehensive understanding of the company data, each data entry or item would need to be individually read and analyzed.

Thus, there is a need in the art for search systems and methods and data analysis systems and methods that capture the meaning of documents and data, and convey that deeper understanding to the user. More particularly, there is a need for systems and methods that quantify meaning from data or search results and display it in a usable way to, for example, consumers, marketers, and internet users.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present disclosure, in one embodiment, relates to a system for analyzing and presenting data. The system may include a mapping engine in communication with an information database storing a plurality of records. The mapping engine may create a map defining a value, category, or core trend from at least a first record by applying a text mining tool to the first record. The mapping engine may determine whether a second record relates to the value, category, or core trend by comparing the second record to the map using a semantics analysis. Moreover, the mapping engine may assign a score to the second record based on the semantics analysis. The score may signal how well the second record relates to the value, category, or core trend. The mapping engine may additionally associate the second record with the value, category, or core trend based on the score. In some embodiments, the system may additionally have an analysis module in data communication with the mapping engine. The analysis module may create graphic visualizations of records associated with the value, category, or core trend, and may present the graphic visualizations via a user interface. In some embodiments, associating the second record with the value, category, or core trend may include comparing the score to a threshold, and, if the score meets or exceeds the threshold, associating the second record with the value, category, or core trend. The system may have a custom collections database in some embodiments. The custom collections database may have a plurality of records from which the mapping engine creates the map. The custom collections database may additionally have a values library, a categories library, and a core trends library. In some embodiments, the mapping engine may store the map in the custom collections database. In some embodiments, the system may have a clustering engine in data communication with the information database. The clustering engine may derive a concept and/or cluster from the second document. The mapping engine may create a map for each of a plurality of values, a plurality of categories, and a plurality of core trends, in some embodiments. The user interface may be configured to receive a search term entered by a user for searching the information database. Moreover, the second record may be a search result from a search of the information database in some embodiments.

The present disclosure, in another embodiment, relates to a method for retrieving and interpreting search results from a search of an information database storing a plurality of records. The method may include creating a map defining a value, category, or core trend, receiving a search term at a user interface, searching the information database for the search term to generate a plurality of search results, determining whether the search results relate to the value, category, or core trend by comparing each of the search results to the map using a semantics analysis. Moreover, the method may include assigning a score to each search result based on the semantics analysis. The score may signal how well the search result relates to the value, category, or core trend. The method may include associating at least one of the search results with the value, category, or core trend based on the assigned scores. The method may additionally include presenting the search results, at the user interface, based on associated concepts, clusters, values, categories, and/or core trends. The step of creating a map may include applying a text mining tool to a plurality of records from the information database. Moreover, the method may include deriving concepts and/or clusters from the search results using a clustering engine. In some embodiments, the method may include storing the map in a custom collections database comprising a plurality of records from which the mapping engine creates the map. The custom collections database may additionally have a values library, a categories library, and a core trends library. In some embodiments, the method may include creating a map for each of a plurality of values, a plurality of categories, and a plurality of core trends.

The present disclosure, in another embodiment, relates to a method of analyzing a plurality of data records. The method may include receiving the plurality of data records, creating a map defining a value, category, or core trend, and determining whether the data record relates to the value, category, or core trend by comparing each data record to the map using a semantics analysis. Moreover, the method may include assigning a score to each data record based on the semantics analysis. The score may signal how well the data record relates to the value, category, or core trend. The method may include associating at least one of the data records with the value, category or core trend based on the assigned scores. The method may additionally include presenting the data records, at the user interface, based on associated concepts, clusters, values, categories, and/or core trends. The step of creating a map may include applying a text mining tool to a plurality of data records. In some embodiments, the method may include deriving concepts and/or clusters from the data records using a clustering engine. The method may include storing the map in a custom collections database storing a plurality of records from which the mapping engine creates the map. In some embodiments, the method may include creating a map for each of a plurality of values, a plurality of categories, and a plurality of core trends.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with Figures that are displayed within the text below of this provisional application.

FIG. 1 is a schematic of a system of the present disclosure, according to one or more embodiments.

FIG. 2 is a schematic of another system of the present disclosure, according to one or more embodiments.

FIG. 3 is a schematic of another system of the present disclosure, according to one or more embodiments.

FIG. 4 is a schematic of another system of the present disclosure, according to one or more embodiments.

FIG. 5 is a flow diagram of a method of the present disclosure, according to one or more embodiments.

FIG. 6 is an example of a user interface of the present disclosure displaying a search bar, according to one or more embodiments.

FIG. 7 is an example of a user interface of the present disclosure displaying search results, according to one or more embodiments.

FIG. 8 is an example of a user interface of the present disclosure displaying a data sphere, according to one or more embodiments.

FIG. 9 is an example of a user interface of the present disclosure displaying a concept wheel, according to one or more embodiments.

FIG. 10 is an example of a user interface of the present disclosure displaying a cluster display, according to one or more embodiments.

FIG. 11 is an example of a user interface of the present disclosure displaying a categories view, according to one or more embodiments.

FIG. 12 is an example of a user interface of the present disclosure displaying a values view, according to one or more embodiments.

FIG. 13 is an example of a user interface of the present disclosure displaying a spending by category view, according to one or more embodiments.

FIG. 14 is an example of a user interface of the present disclosure displaying a core trends view, according to one or more embodiments.

FIG. 15 is an example of a user interface of the present disclosure displaying a momentum view, according to one or more embodiments.

FIG. 16 is an example of a user interface of the present disclosure displaying a visual gallery, according to one or more embodiments.

FIG. 17 is an example of a user interface of the present disclosure displaying a big picture analysis, according to one or more embodiments.

FIG. 18 is an example of a user interface of the present disclosure displaying a dashboard view, according to one or more embodiments.

FIG. 19 shows a plotting map of the present disclosure, according to one or more embodiments.

FIG. 20 is a flow diagram of another method of the present disclosure, according to one or more embodiments.

FIG. 21 is a chart of values found within customer reviews of a product, according to one or more embodiments.

FIG. 22 is a chart of values tracked over time with respect to customer reviews of a product, according to one or more embodiments.

FIG. 23 is a close-up view of a portion of the chart of FIG. 22.

### DETAILED DESCRIPTION

The present disclosure relates to novel and advantageous systems and methods for analyzing data. Particularly, the present disclosure relates to systems and methods for analyzing a plurality of data records to provide a comprehensive understanding of the data. For example, in some embodiments, systems and methods of the present disclosure relate to searching one or more public or private databases based on a user's search term(s). The results from the search may be analyzed to determine values, categories, and/or core trends within the search results. The values, categories, and/or core trends may be pre-defined, and in some embodiments may be customized based on user needs. The search results may additionally or alternatively be analyzed to determine concepts and/or clusters present within the search results. The search results may be grouped or organized based on the values, categories, core trends, clusters, and/or concepts, and may be presented to a user via a user interface. The user interface may provide a comprehensive understanding of the search results, by graphically illustrating the search results according to the values, categories, core trends, concepts, and/or clusters. Additionally, in some embodiments, systems and methods of the present disclosure relate to analyzing public or private company data, and tracking or monitoring the data over time. For example, customer reviews, complaints, social media posts, or other data related to a company or product may be analyzed to determine values, categories, core trends, concepts, and/or clusters. One or more databases may be monitored, such that new reviews, complaints, posts, or other data entries may be received and analyzed in real time, substantially real time, periodically, or at any other interval. A user dashboard may provide a graphic display of real time analysis of the company data, according to values, categories, core trends, concepts, and/or clusters. As used herein, “real time” may generally refer to at or substantially near the same time. For example, where data or search results are received for analysis, the data or search results may be analyzed, and results of the analysis may be made available to a user, substantially near the time the data or search results are received

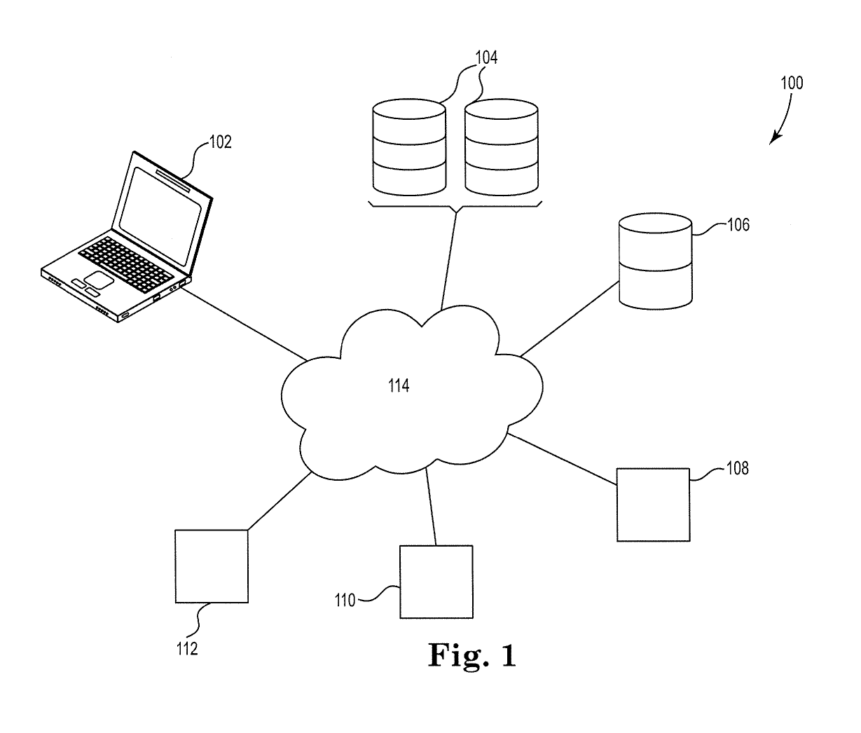

Turning now to FIG. 1, a concept analysis system 100 of the present disclosure is shown, according to one or more embodiments. The concept analysis system 100 may generally have a user interface 102, one or more information databases 104, a custom collection database 106, a mapping engine 108, a clustering engine 110, and an analysis module 112. The user interface 102, information database(s) 104, custom collection database 106, mapping engine 108, clustering engine 110, and analysis module 112 may generally be connected over a wired or wireless network 114. In some embodiments, the network 114 may be a local area network. In other embodiments, the network 114 may be an Internet network. In some embodiments, the components of the system may communicate over more than one network. For example, one database 104 may be connected over a local area network, while another database may be accessible over an Internet network. In some embodiments, the system 100 may perform, or facilitate performance of, a search to find relevant documents or data entries. The search may be performed based on, for example, one or more search terms entered by a user at the user interface 102. The system 100 may analyze and/or organize the search results based on various parameters, as described below. The analysis may be performed in real time in some embodiments. The system 100 may generally be agnostic to the type of data or type of database(s) searched, such that generally any suitable data records may be analyzed in accordance with the systems and methods described herein. However, in some embodiments, analysis of the data records may be performed based on custom or tailored parameters.

The user interface 102 may generally allow a user to access the various components of the system 100. The user interface 102 may allow a user to input information, as well as view and modify information. The user interface 102 may be provided via a desktop computer, laptop computer, tablet computer, smartphone, or any other suitable computing device. In some embodiments, the user interface 102 may be a website. In other embodiments, the user interface 102 may be a computer program. In some embodiments, the user interface 102 may provide a login page wherein a user may input a username and password, for example, to log into the system 100. In some embodiments, analyses performed by the system 100 may be conducted based on information input by a user at the user interface 102. For example, a user may input a search term for searching the one or more information databases 104 via the user interface 102. Moreover, the results of analyses performed by the system 100 may be displayed at the user interface 102. For example, the user interface 102 may provide a real time dashboard showing up-to-date analyses performed by the system 100.

The information database(s) 104 may generally comprise data that is accessible and/or searchable by the mapping engine 108, clustering engine 110, and/or analysis module 112. One or more information databases 104 may comprise public data, such as articles, blogs, videos, webpages, social media posts, government data, trusted data collections, and/or websites. Additionally or alternatively, one or more information databases 104 may comprise client data, which may include client-specific data including marketing data, product information, or research and development information, which may or may not be private data. Each information database 104 may be local or remote to other system components.

The custom collection database 106 may comprise stored data, such as articles, documents, webpages, social media posts, etc., or extrapolated data therefrom, for at least one of a search topic, a core trend, a category, a value, or a concept. The documents and data in the custom collection database 106 may be derived from the one or more information databases 104. The custom collection database 106 may collect and store relevant data or documents, from the one or more information databases 104, for each core trend, value, category, or concept. For example, the system 100 may collect the 1,000, or other suitable number, data entries or documents most related to a particular value, and then store those relevant entries or documents for the particular value within the custom collection database 106. In at least one embodiment, the custom collection database 106 may have one or more relevant data entries or documents for each core trend, value, category, and/or concept of the mapping engine 108. In some embodiments, the documents or data stored for each trend, value, category, or concept may be selected manually or partially manually by a user, for example. In other embodiments, the documents or data stored for each trend, value, category, or concept may be selected automatically by, for example, the mapping engine 108. In some embodiments, the system 100 may continually, periodically, or randomly search the one or more information databases 104 to change or increase the stored data in the custom collection database 106. This stored data or documents within the custom collection database 106 may be retrievable by at least the mapping engine 108, as described below.

The mapping engine 108 may generally be configured to determine correlations between data in the information database(s) 104 and one or more values, one or more categories, and/or one or more core trends.

A “value” may be defined in some embodiments as a criterion of importance or significance (for example, authenticity, safety, fear, power, freshness, health, etc.). Values may have positive, negative, or neutral connotations. Values may include for example, but are not limited to, Access, Achievement, Adventure, Affluence, Ambition, Aspiration, Assistance, Authenticity, Balance, Beauty, Belief, Belonging, Celebration, Challenge, Change, Choice, Civility, Comfort, Commitment, Community, Compassion, Competition, Confidence, Connectivity, Conservation, Contentment, Control, Convenience, Cool, Courage, Creativity, Curiosity, Deliciousness, Dependability, Design, Desire, Detail, Devotion, Dignity, Discipline, Discovery, Diversity, Efficiency, Empowerment, Endurance, Energy, Entertainment, Equality, Escape, Excellence, Exclusivity, Experience, Expertise, Faith, Family, Fantasy, Fear, Fitness, Fragility, Freedom, Fresh, Friendship, Fun, Future, Growth, Happiness, Harmony, Health, Honesty, Honor, Hope, Idealism, Identity, Image, Independence, Individuality, Indulgence, Information, Ingenuity, Innovation, Inspiration, Integrity, Intelligence, Intimacy, Intuition, Joy, Justice, Knowledge, Leadership, Learning, Legacy, Logic, Love, Loyalty, Luck, Luxury, Maturity, Nature, Natural, Nostalgia, Novelty, Optimism, Order, Originality, Passion, Patience, Patriotism, Peace, Performance, Personalization, Pleasure, Populism, Power, Prevention, Pride, Privacy, Prosperity, Purity, Quality, Relaxation, Respect, Responsibility, Romance, Safety, Security, Sensuality, Serenity, Sexuality, Sharing, Simplicity, Speed, Spirituality, Stability, Status, Stealth, Strength, Style, Subversion, Success, Sustainability, Teamwork, Thrift, Thrill, Transparency, Trust, Truth, Uniqueness, Unity, Value, Vitality, Wealth, Wellness, Whimsy, Wisdom, and Wonder.

A “category” may be defined as a classification of similar things (for example, sports, health & beauty, fashion, home furnishings, automotive, manufacturing). Categories may include for example, but are not limited to, categories used or defined by the standard categories of the Open Directory Listing Project (ODLP), subsets thereof, or any other defined category of textual meaning. In some embodiments, categories may be derived from the Bureau of Labor Statistics (BLS). A category in this system 100 may also refer to sub-categories within a broader defined category. The system 100 may also comprise tiered categories of multiple categories, wherein each category has a different level of scope or importance. In some embodiments, a category may also be a value (for example, safety). The mapping engine 108 may define a finite number of values and/or categories. In at least one embodiment, the mapping engine 108 may be in data communication with the user interface 102 such that the user may select the at least one value and/or category that the user wishes to measure or compare with respect to the data.

A “core trend” may be societal or cultural metrics that may help to indicate where a document or data entry coincides with one or more forces of the consumer or cultural landscape. For example, core trends may include, but are not limited to society, technology, economy, environment, and politics (i.e., “STEEP”). In other embodiments, core trends may be any other suitable societal or cultural metrics. Core trends may be selectable by a user in some embodiments.

In some embodiments, the mapping engine 108 may have a categories library, a values library, and/or a core trends library. For example, a categories library may comprise a number of categories, which in some embodiments may be derived from categories used or defined by the standard categories of the Open Directory Listing Project (ODLP), any other suitable category listing, or subsets thereof. In some embodiments, the system 100 may use an algorithm such as Naïve Bayes Classifier or another algorithm to assist with categorization. The custom collection database 106 may include multiple data entries (documents, articles, social media posts, and other data) associated for each category of the categories library. For example, the custom collection database 106 may comprise up to 500, or other suitable number, or more relevant documents for each category of the categories library. Using a text mining tool such as open source NLP utility LingPipe, Apache Mahout, Mallet, or similar tool, the mapping engine may create a map for one or more categories in the category library. A map may generally be or include an association between a category and documents in the categories library. In one embodiment, the map for a category may include the particular documents in the categories library that correspond with, or have been found via the text mining tool or another tool to relate to, the category. In another embodiment, the map for a category may include a numerical value or score, or a plurality of numerical values or scores, which may be produced by one or more algorithms. The maps created for each category may be stored in the custom collection database 106. In at least one embodiment, the mapping engine 108 has at least one stored map in the custom collection database 106 for each category.

In at least one embodiment, a Categories library may be pre-loaded with a number of categories, which may have up to three tiered levels or sub-levels: a broad category, zero, one, or more sub-categories of each broad category, and zero, one, or more sub-categories of each sub-category. In one example embodiment, the Categories library may have up to 755, up to 770 categories, or more categories. The system 100 may then retrieve, for example, as many as up to 500 or more relevant results for each of the categories of the Categories library and store these category results in the customs collection database 106. Using an NLP utility such as Apache Open NLP, LingPipe, Apache Mahout, Mallet, or other tool, a map of the data is created that characterizes the content for each category and then stores that category map in the customs collection database 106.

The mapping engine 108 in some embodiments may similarly comprise a values library comprising a number of values, and may map the values to documents in the custom collection database 106. The custom collection database 106 may include stored articles, documents, etc., or other data associated for each value of the values library. For example, the custom collection database 106 may comprise up to 500, or other suitable number, or more relevant documents for each value of the categories values. Using a text mining tool such as Apache Open NLP, LingPipe, Apache Mahout, Mallet, or similar tool, the mapping engine 108 may create a value map for each value to the data in the custom collection database 106. A map may generally be or include an association between a value and documents in the values library. In one embodiment, the map for a value may include the particular documents in the values library that correspond with, or have been found via the text mining tool or another tool to relate to, the value. In another embodiment, the map for a value may include a numerical value or score, or a plurality of numerical values or scores, which may be produced by one or more algorithms. In at least one embodiment, the mapping engine 108 has at least one stored value map in the custom collections database for each value.

In some embodiments, the mapping engine 108 may similarly have a core trends library comprising a number of core trends and a number of values and/or categories associated with each core trend. For example, each of the STEEP core trends may be associated with a number of values. The mapping engine may additionally map the core trends to documents in the custom collection database 106. For example, the custom collection database 106 may comprise up to 500, or other suitable number, or more relevant documents for each value associated with at least one of the core trends. Using a text mining tool such as open source NLP utility LingPipe, Apache Mahout, Mallet, or similar tool, a core trend map can be generated from the data for each core trend and then stored. A map may generally be or include an association between a core trend and documents in the core trends library. In one embodiment, the map for a core trend may include the particular documents in the core trends library that correspond with, or have been found via the text mining tool or another tool to relate to, the core trend. In another embodiment, the map for a core trend may include a numerical value or score, or a plurality of numerical values or scores, which may be produced by one or more algorithms. In at least one embodiment, the mapping engine 108 has stored core trend maps in the custom collection database 106 for each core trend.

The mapping engine 108 may additionally be configured to map documents or data entries in the one or more information databases 104. Specifically, in some embodiments, the mapping engine 108 may be configured to determine one or more scores for one or more data entries or documents in the information database(s) 104. A score may be determined for a particular data entry or document by comparing the document to a value, category, or core trend map provided by the mapping engine 108. In some embodiments, a score may provide an indication of the degree of correlation between a value, category, or core trend, and a data entry or document in a database. For example, a score may be or include a numerical value in some embodiments, signifying or quantifying the degree or amount of correlation between the value, category, or core trend, and the data entry or document. Each data entry or document may correspond with a plurality of scores for a plurality of values, categories, or core trends. In some embodiments, the score may be determined based on a textual analysis and/or linguistic technology used to help quantify the text or meaning in a document or data entry. A semantic analysis may be performed to compare each data entry or document to each value, category, and core trend map. In at least one embodiment of this system 100, however, the Metadata, the content data, or any of the data (e.g., documents, articles, social media posts, etc.) in the information database(s) 104 may be compared against and/or matched with the documents or maps stored in the custom collection database 106. This matching can be done with any existing linguistic technology. By matching the data entries or documents in the one or more databases 104 to the stored documents or maps in the custom collection database 106 via the mapping engine 108, it can be determined which values, categories, and core trends are most or least relevant to each data entry or document in the information database(s).

In some embodiments, the system 100 may additionally include a clustering engine 110, such as Carrot2, a clustering engine that uses the Lingo or STC Clustering Algorithm, LingPipe, another open source clustering engine, or any other suitable clustering engine. The clustering engine 110 may be configured to determine concepts and/or clusters present in the data entries or documents in the information database(s) 104. In one embodiment, “concepts” are words or phrases that identify the prominent subject represented in a data entry or document, such as an article, blog, social media post, etc. A “cluster” may be a collection of words that appear frequently across a plurality of documents or data entries.

The analysis module 112 may generally compile and/or analyze information received from the mapping engine 108 and clustering engine 110. In some embodiments, the analysis module 112 may provide an output, such as a display or a report, to present data from the informational database(s) 104 to a user according to the values, categories, core trends, concepts, and/or clusters. In some embodiments, the analysis module 112 may provide relevant document or data entries, or links thereto, from the information database(s) 104. As indicated above, for a given set of data or search results the analysis module 112 can identify and display top matching categories, top matching values, and matching core trends for the search results as a whole. In further embodiments, relationships may be created or analyzed between the resulting categories, values, and/or core trends. For example only, after identifying the top matching categories, the analysis module may identify the data entries, search results, documents, etc. that match the top matching categories or a subset thereof. Then, the analysis module 112 can identify the values and/or core trends associated with those entries or documents to create or identify relationships or associations between the categories and the values and/or core trends, thereby using the data entries or documents to link categories, values, and/or core trends and see how these are aligned with one another. The foregoing is just one example. Of course this type of relationship analysis could alternatively start with the data entries or documents that match the top matching values or a subset thereof (or that match one or more specific core trends). Then, the analysis module 112 can identify the categories and/or core trends (or categories and/or values) associated with those data entries or documents to create or identify relationships or associations between the values and the categories and/or core trends (or between the core trends and the categories and/or values).

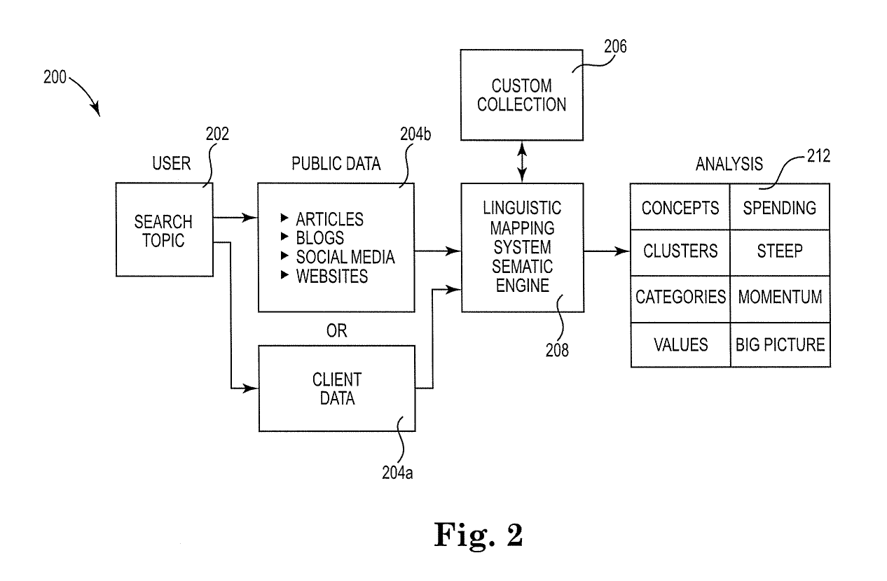

FIG. 2 shows another system 200 of the present disclosure. The system 200 may include a user interface 202 wherein a user may input a search topic, for example. The search topic may be used to search private client data 204a, and/or public data 204b, such as Internet websites, articles, social media, blogs, and/or other data. However, in other embodiments, documents or data entries in a public or private data set may be analyzed without first being searched. A mapping engine/clustering engine 208 may compare the private and/or public data with data stored in a custom collection database 206 in order to, for example, correlate the public and/or private data with one or more values and/or categories, and to determine from the data one or more clusters and/or concepts. Moreover, an analysis module 212 may compile and analyze information received from the mapping engine/clustering engine 208 and present the information to a user via the user interface 202.

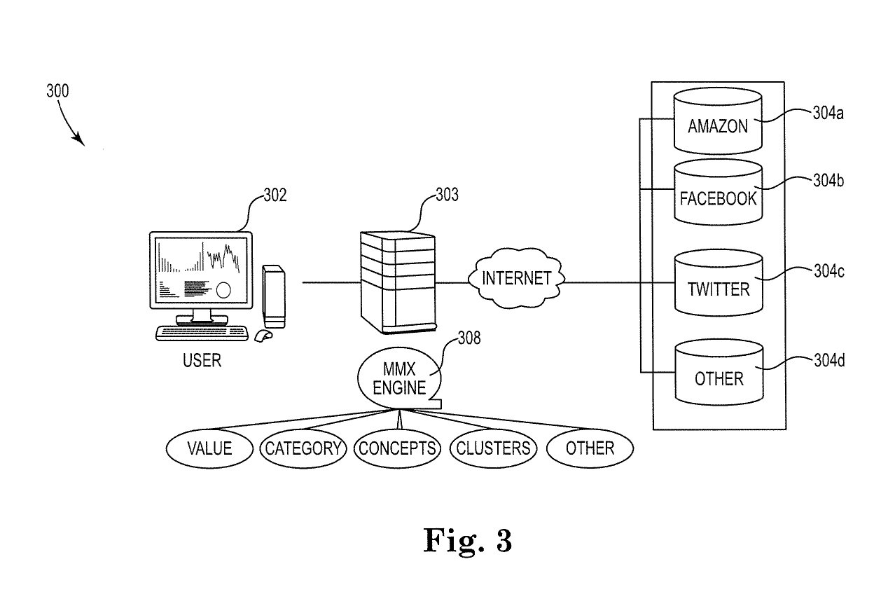

FIG. 3 shows another system 300 of the present disclosure. The system 300 may have a user interface 302 whereby a user may access a server 303. The server 303 may have hardware and/or software that provide a mapping and clustering engine/analysis module 308. In some embodiments, the server 303 may be, or may include, a general purpose computer or general purpose components configured for a particular purpose. The server 303 may access one or more databases, such as an Amazon database 304a, a Facebook database 304b, a Twitter database 304c, a consumer's or company's own private database, and/or other public and/or private databases 304d. In some embodiments, the server 303 may access these databases over the Internet, or another wired or wireless network. The mapping and clustering engine/analysis module 308 may compare data in the one or more databases 304 to one or more values and/or categories, and may determine from the data one or more concepts and/or clusters. The mapping engine/analysis module 308 may further analyze and/or present the data, in terms of the values, categories, concepts, and/or clusters, via the user interface 302.

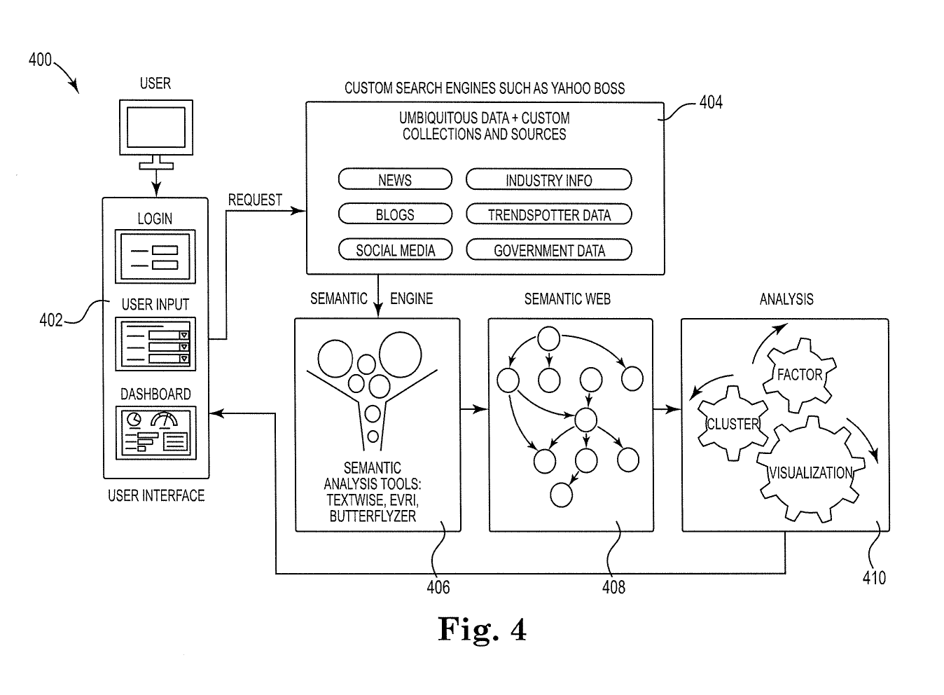

FIG. 4 shows another system 400 of the present disclosure. The system 400 may have a user interface 402, whereby a user may enter login information and a user input, such as one or more search terms. A search engine 404 may use the search term(s) to search public news, blogs, social media, industry information, trendspotter data, government data, and/or other public or private databases. The search engine 404 may retrieve search results, which may be sent to a semantic engine 406. The semantic engine 406, which may be or include TextWise, Butterflyzer, or any other suitable semantic tool, may evaluate the search results to extract meaning from the relevant content. In some embodiments, the semantic engine 406 may produce a semantic web 408 of concepts, categories, values, core trends, or signatures found within the search results. An analysis module 410 may analyze the semantic web 408 and/or search results for mapping and analysis of trends, meaning, and intelligence. Additionally, the analysis engine 410 may perform a set of processing steps to interpret the information retrieved, including but not limited to factor analysis and correlation analysis. A factor analysis or correlation analysis may apply one or more statistical algorithms to the determined concepts, categories, values, core trends, and/or signatures to find alignment among them. For example, where one or more search results correlates with the values of “fresh” and “natural,” factor analysis and/or correlation analysis may find or identify a correlation between those two values and the related search results.

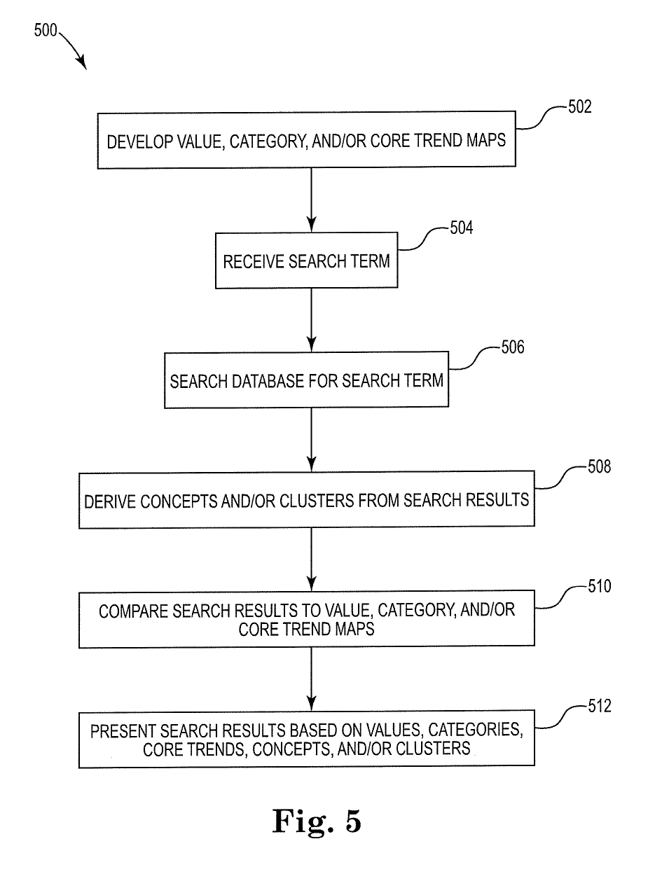

Systems of the present disclosure may be configured to perform one or more methods. For example, FIG. 5 shows a method 500 for searching one or more public or private databases and providing meaningful search results, performable by one or more systems of the present disclosure, according to one embodiment. The method 500 may include developing value, category, and/or core trend maps 502; receiving a search term 504; searching a database for the search term 506; deriving concepts and/or clusters from the search results 508; comparing the search results to the value, category, and/or core trend maps 510; and presenting the search results with meaning based on values, categories, core trends, concepts, and/or clusters 512. In other embodiments, the method 500 may include additional and/or alternative steps. Moreover, these steps may be performed in any suitable order, and are not limited to the order shown in FIG. 5.

As indicated above, the method 500 may include developing value, category, and/or core trend maps 502. As described above, values, categories, and/or core trends may be words or phrases that may convey an idea, feeling, or other parameter. The maps may be developed based on documents in the custom collections database, for example, which may be selected based on their relevance to a particular value, category, or core trend. In some embodiments, a map may include a series of words, phrases, or semantic combinations that correlate with the particular value, category, or core trend. In some embodiments, a map may be developed for each value, category, and core trend. The values, categories, and or core trends for which maps are created may be predefined in some embodiments. The values, categories, and/or core trends may be defined by one or more standards, or may be defined by a user or a client, as indicated above.

The method 500 may additionally include receiving a search term 504. The search term may be received from a user, for example. A user may enter or select a search term via a user interface. In other embodiments, a search term may be received from a different source, or may be automatically generated, for example, based on one or more parameters or conditions. The search term may be a word or phrase, for example. Moreover, in some embodiments, multiple search terms may be received and searched simultaneously.

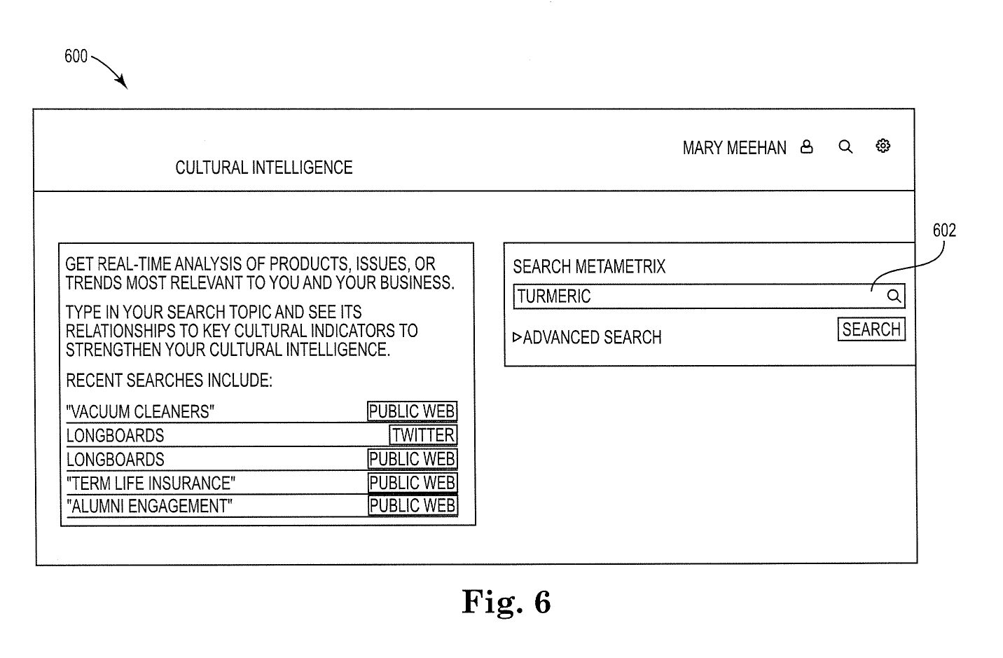

FIG. 6 shows one embodiment of a user interface 600 whereby a user may enter the search term. The interface 600 may provide a search box 602 wherein a user may type or otherwise enter a search term. As an example shown herein to illustrate systems and methods of the present disclosure, a search term may be “turmeric,” for example, as in FIGS. 6-18. In some embodiments, the user may have options to restrict the search to one or more particular values, categories, clusters, or concepts. Additionally or alternatively, other options may allow a user to restrict the search to one or more particular databases, geographic regions, languages, time periods, and/or demographic characteristics.

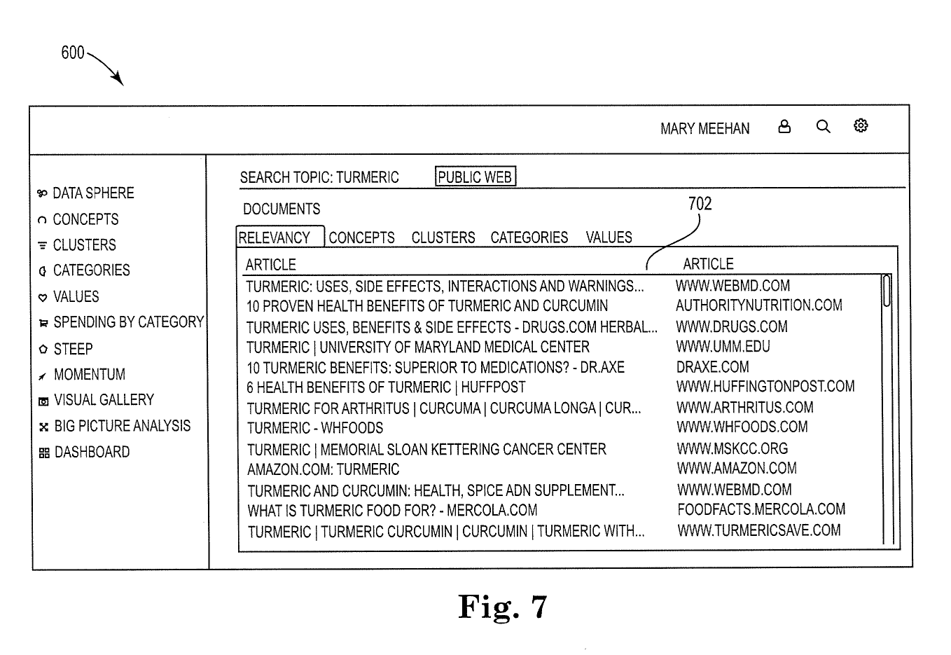

Turning back to FIG. 5, after a search term is received, such as from a user via the user interface, a system of the present disclosure may search one or more databases for the search term 506. In this way, the search may be similar to other Internet searches performed by Internet search engines, where the system may return a list of results that contain the search term or contain terms that are similar or related terms to the search term. The system may search websites, news articles, blogs, social media, and/or other public or private data accessible via the Internet or any other suitable network. The search results may be or include a list of articles, documents, links, or other data entries. In some embodiments, the search results may be displayed or viewable via the user interface. For example, FIG. 7 shows one embodiment of a list of search results for the search term “turmeric,” as a list of documents 702. The search results may be provided or organized according to any suitable organization. For example, as shown in FIG. 7, the results may be provided in order of relevancy. Relevancy may be determined based on the number of times the search term appears in the document, the prominence of the search term, and/or any other suitable characteristics. In some embodiments, a user may have the option to view the search results according to other organization. For example, the user may view the search results ordered by associated concepts, clusters, categories, or values, as will be more fully described herein.

The method 500 may include deriving concepts and/or clusters from the search results 508. As described above, for example, a clustering tool such as Carrot2, a clustering engine that uses the Lingo and STC Clustering Algorithms, LingPipe, another open source clustering engine, or any other suitable clustering tool may be used to derive concepts and clusters from the search results. In some embodiments, the search results may be organized or grouped by clusters and/or concepts.

The method 500 may include comparing the search results to the value, category, and/or core trend maps to determine correlation between the search results and the values, categories, and core trends 510. As indicated above, a mapping engine, for example, may compare each search result to each value, category, and core trend map. Semantics tools may be used to compare the search results to the maps to determine a correlation between each search result and each map.

In some embodiments, comparing the search results to the value, category, and/or core trend maps may include assigning a score to each search result with respect to each value, category, and/or core trend. For example, each document may be assigned a score with respect to the value of energy. The score may be determined by comparing each document with the map for the value of energy. Algorithms such as, but not limited to, Naïve Bayes may be used to compare the document with the map to produce a numbered score, for example. The score may signal how strongly the document correlates with the value of energy. In some embodiments, a higher score may signal a stronger correlation with the value, category, or core trend, whereas a lower score may signal a weaker correlation with the value, category, or core trend. In one embodiment, if there is no evidence of a correlation between a particular search result and a particular value, category, or core trend, the search result may receive a score of zero for that particular value, category, or core trend. In another embodiment, a clear correlation between a document and a map may produce a score of zero, and lesser correlations or a lack of correlation may result in negative scores. Each search result or document may thus be assigned multiple scores, one for each value, category, and/or core trend. In some embodiments, a threshold may be applied to the value, category, and/or core trend scores for the search results. A threshold may determine whether each search result will be presented as associated with a particular value, category, or core trend. If a document meets or exceeds the threshold for the value of energy, for example, the document may be associated with the value of energy. In some embodiments, the scores assigned to a search result or document may be ordered, such that the value, category, and/or core trend having the highest correlation to the document may be selected as a predominant or most relevant value, category, or core trend for the search result.

In at least one embodiment, each relevant result may be limited to matching with a maximum number of values, categories, and/or core trends, which may be a pre-selected or pre-configured number, and may be configurable by a user. For example, in one embodiment, each relevant result may be matched with a maximum of five categories and five values. For even further example, where the relevant results from a user's search amount to 100 results and the maximum number of categories that each result may be matched with is five categories, there will be a maximum of 500 category data points for the user's search result. Once matched, the categories analysis can be displayed visually in various forms that show the relative importance or ranking of the categories matched with the search results.

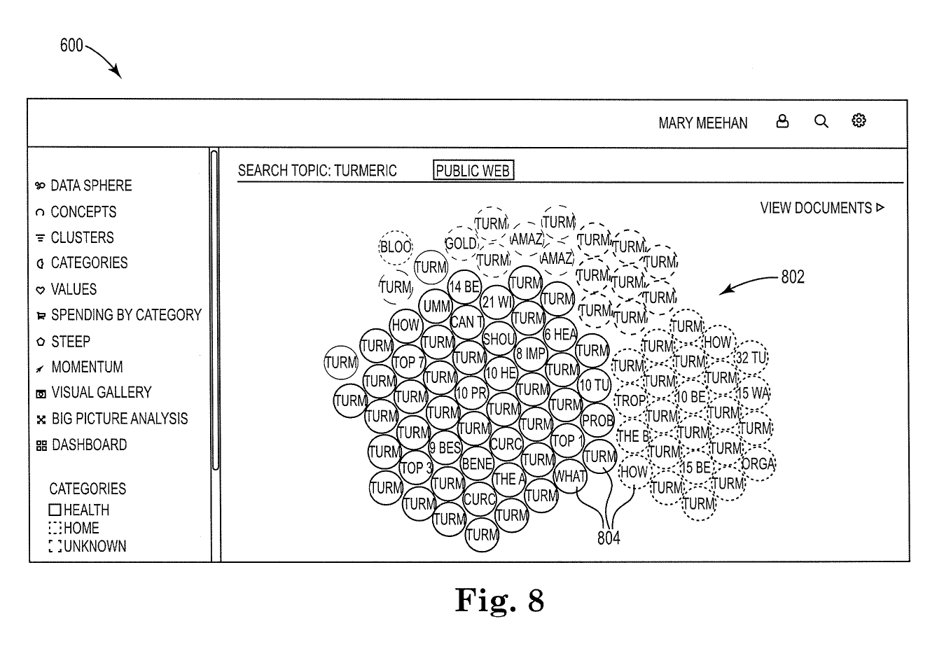

The method 500 may additionally include presenting the search results to a user 512. In some embodiments, the search results may be presented via a dynamic, interactive user interface. In other embodiments, the search results may be presented via a report or other output. FIGS. 8-18 show one embodiment of a user interface 600 presenting search results for the search term turmeric. The search results may generally be presented according to the values, categories, clusters, and/or concepts. The search results may also be presented in terms of trends and other organizational tools. As described with respect to FIGS. 8-18, in some embodiments, the search results may be presented using a data sphere, a concept wheel, a cluster display, a categories display, a values display, a spending display, a core trends analysis, a momentum analysis, a visual gallery, a big picture analysis, and a dashboard display.

FIG. 8 shows one example of a data sphere 802, which may be a visual display of at least some of the search results. The data sphere 802 may provide a visual grouping of at least a portion of the search results based on assigned categories, for example. In at least one embodiment, each result (e.g. article, webpage, social media post, etc.) may be displayed as an individual icon 804. In FIG. 8, the icon 804 for each search result is shown as a circle or a bubble, but in other embodiments it may be a square, a triangle, or have some other configuration. In at least one embodiment, the icon 804 may be colored a particular color based on a predominant category or value of the search result. For example, the search results having a predominant assigned category of health may be shown as blue circles, and the search results having an assigned predominant category of home may be presented as orange circles. In some embodiments, the color-coded icons 804 may be or include links to the search results, such that a user may select any one of the icons and the associated search result may be displayed. In this way, the user may see visually what categories are associated with the results for the search topic and may access those results that are pertinent to one category based on the color coded display. In other embodiments, the data sphere 802 may group and color result icons based on predominant values. In still other embodiments, the data sphere 802 may group and color result icons based on categories or clusters.

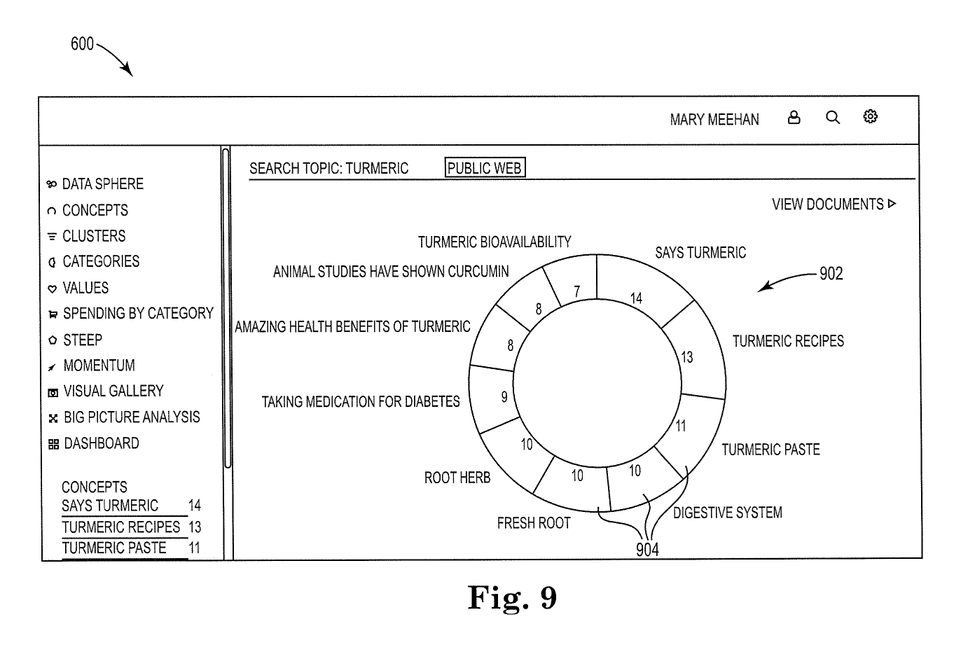

FIG. 9 shows one example of a concept wheel 902, which graphically presents the concepts found in the search results. In some embodiments, the concept wheel 902 may graphically represent the top ten, or any other suitable number, of most relevant concepts. In at least one embodiment, the concept wheel 902 graphically presents the concepts as a part of a pie-like graph, where the size and/or the color of each section 904 of the graph represents the relative relevance of the concept in the search results, or the number of search results exhibiting that concept. For example, as shown in FIG. 9, “Says Turmeric” has the largest slice 904 of the pie-like graph, whereas “Turmeric Bioavailability” has the smallest slice of the pie-like graph. If a user selects an individual slice 904, the search results pertaining to that particular concept may be viewable by the user. In other embodiments, the concepts may be displayed using any other suitable graphic or chart, and is not limited to a wheel display.

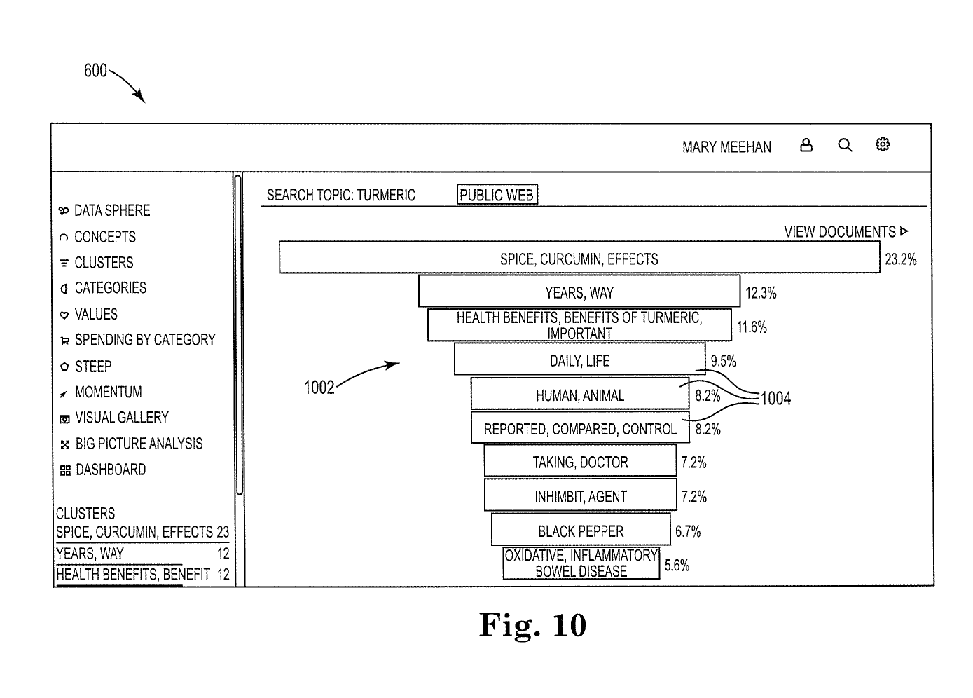

FIG. 10 shows one example of a cluster display 1002, which graphically presents the clusters found in the search results. In some embodiments, the cluster display 1002 may graphically represent the top ten, or any other suitable number, of most relevant clusters. In the cluster display 1002, generally, words of similar relevance to one another may appear together in a row 1004, and different rows may indicate different relevance clusters. Moreover, in one embodiment, the most repeated or most prominent word clusters found in the search results may appear at the top in larger fonts, and less frequent or less prominent clusters may appear at the bottom in decreasing font sizes. In some embodiments, a user may have the option to select a cluster row 1004 to access the documents or search results associated with that cluster.

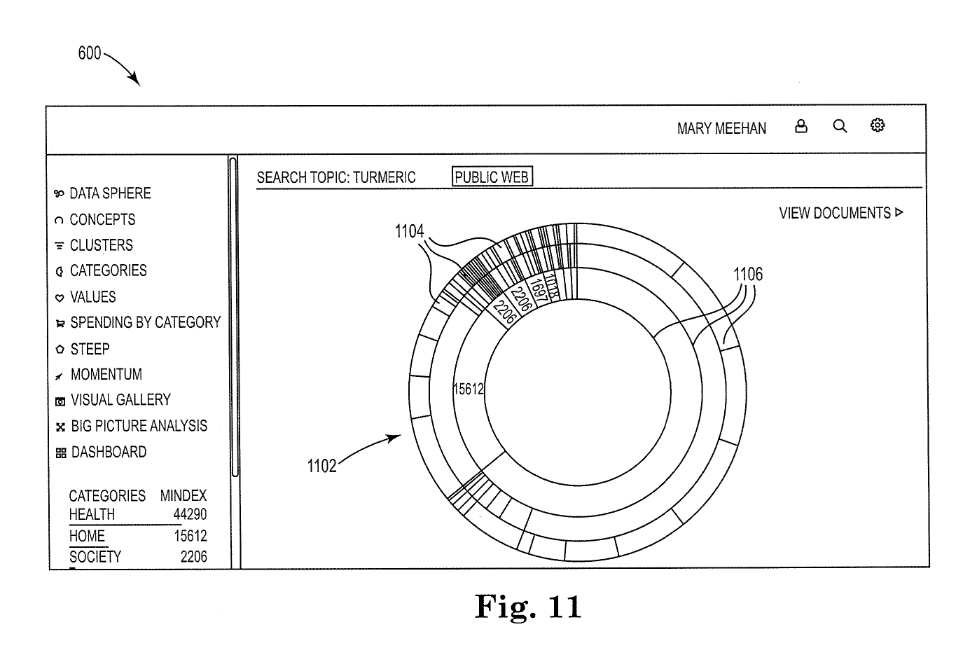

FIG. 11 shows one example of a categories display 1102, which graphically presents the categories found in the search results. As shown in FIG. 11, the categories analysis may be displayed as a pie-like chart with each slice 1104 of the pie-like chart corresponding to a category. The chart may further comprise multiple segmented rings 1106 that represent tiered categories or sub-categories, as discussed above. In at least one embodiment, the chart comprises an inner ring that corresponds to the broad categories (e.g. Health), a middle ring that corresponds to sub-categories (e.g. Cooking) within each broad category (e.g. Health), and an outer ring that corresponds to sub-categories (e.g. Organic) of the sub-category (e.g. Cooking). A user can select each segmented area of the chart to display one or more search results related to that category or sub-category.

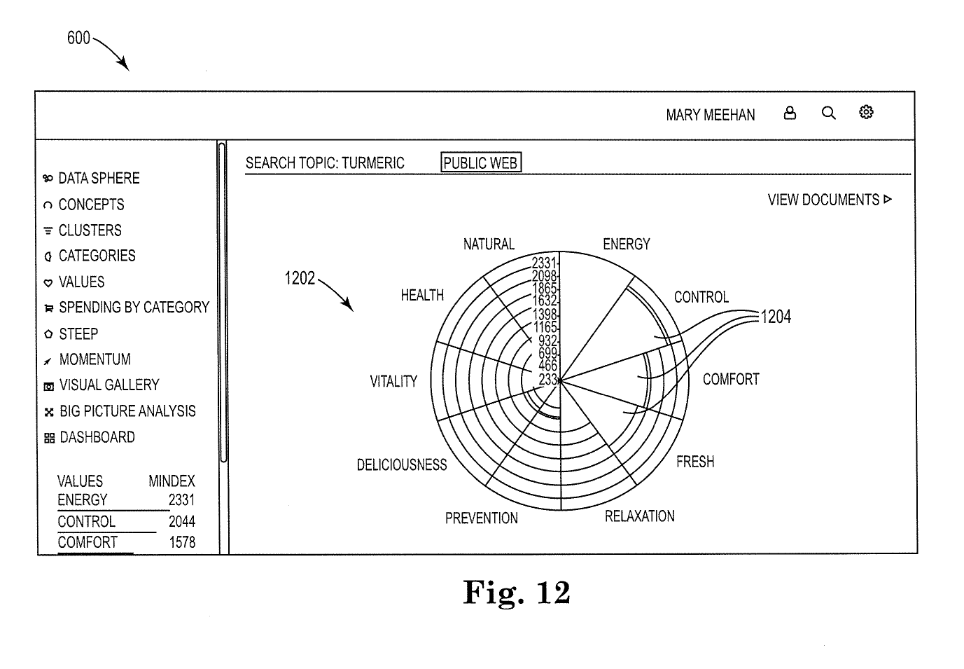

FIG. 12 shows one example of a values display 1202, which graphically presents the values found in the search results. In some embodiments, the values display 1202 may graphically represent the top ten, or any other suitable number, of most relevant values. As shown in FIG. 12, the values display 1202 may be a pie chart comprising a number of color-coded slices 1204 that shows the more relevant values for the search results in a larger slice than the lesser relevant values. The user may select each slice 1204 to display one or more search results related to that value. In other embodiments, the values may be displayed using any other suitable graphic or chart, and is not limited to a pie chart.

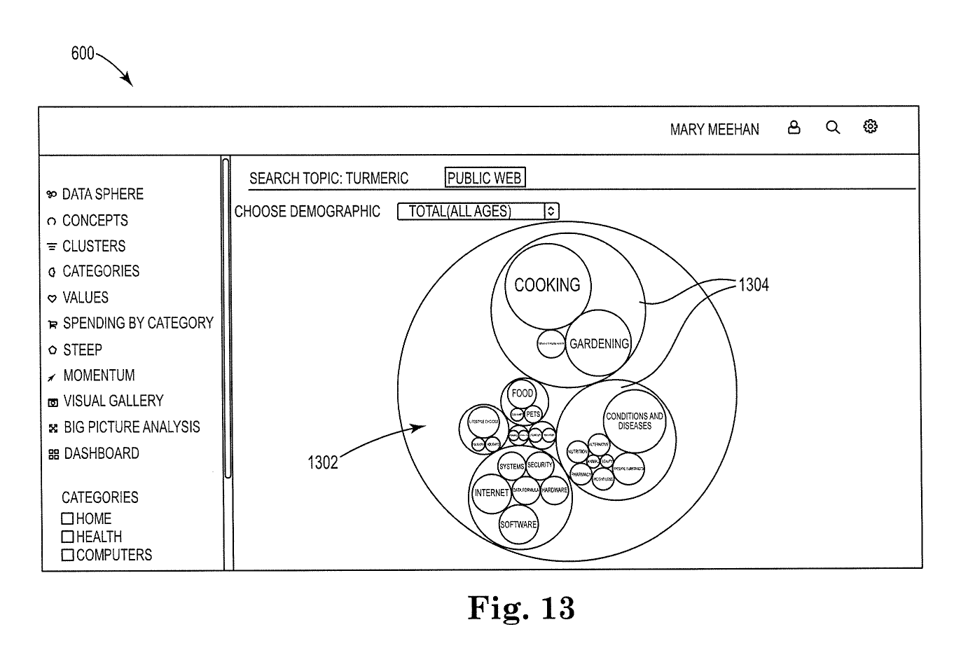

FIG. 13 shows one example of a spending display 1302, which graphically presents one or more categories of the search results with respect to spending data. Spending data may be data retrieved from the Bureau of Labor Statistics (BLS) or other similar data of similar information related to consumer spending. For example, BLS data may be retrieved for tracked spending categories that align with any of the values, categories, and/or core trends. As one particular example, where one or more documents are associated with the category of “business,” BLS spending data for business or other similar or related tracked BLS spending metrics may be presented for the category of “business.” In some embodiments, each value, category, and/or core trend may be mapped to BLS spending data. In some embodiments, the spending data may be presented as a series of color-coded bubbles 1304. Categories and sub-categories may have a same color. Categories may be presented as large bubbles 1304, in which smaller bubbles representing sub-categories may be arranged. In some embodiments, the size of the bubble may correlate with the quantity of spending from the BLS or other spending data. In this way, the smaller bubbles (sub-categories) may identify new, untapped market opportunities. The user may select each bubble of the chart to display relevant results related to that category or sub-category. It may be appreciated that the spending data may additionally or alternatively be correlated with respect to values or core trends, for example.

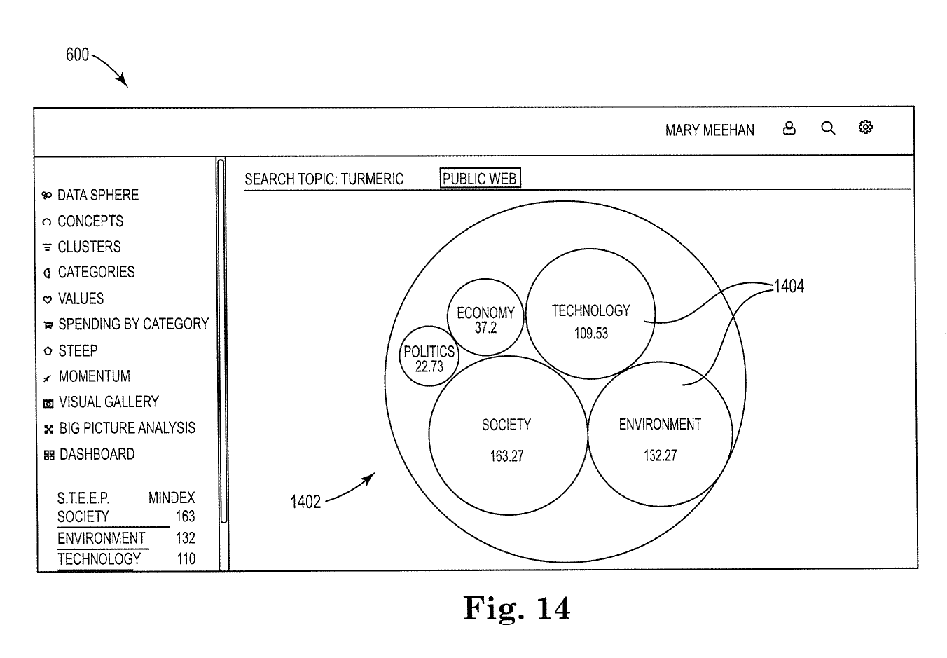

FIG. 14 shows one embodiment of a core trends analysis display 1402, which graphically shows the prominence of core trends in the search results. In some embodiments, each core trend may be displayed as a bubble or icon 1404. In some embodiments, each icon 1404 may have a size correlating with its prevalence in the search results. That is, the larger the display icon 1404 on the core trends analysis display 1402, the greater the match between the search results and the core trend map for the particular core trend. This may generally provide a broader perspective on the consuming landscape.

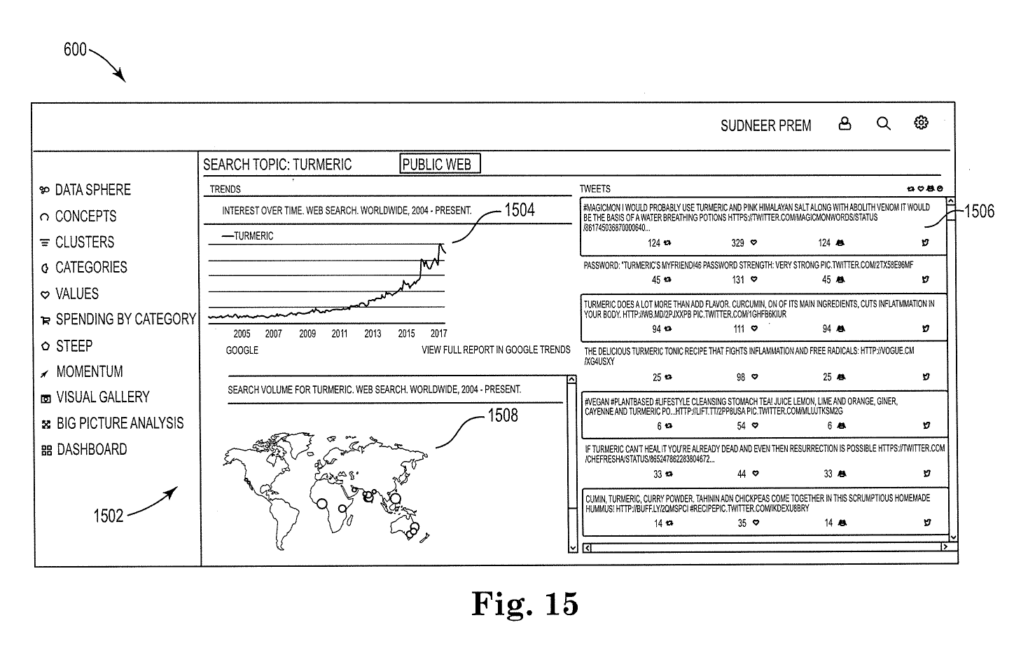

FIG. 15 shows one embodiment of a momentum analysis 1502, which shows trends in searching, number of results over time, engagement over time, and other trend analyses related to the search term. In at least one embodiment, the analysis module may obtain this information from news articles, social media posts, or by using Google Trends or another trend analyzing engine, or similar sources. The momentum analysis may be displayed as a line graph 1504 charting the trend over time. For example, the line graph 1504 may illustrate cultural trend shifts, such as a shift in most important or prevalent value over time. In some embodiments, a line graph may show interest over time, such as number or volume of internet searches or number or volume of relevant documents over time. In some embodiments, most recent Tweets 1506 or other social media posts containing or related to the search term may be displayed. In some embodiments, a map 1508 showing search volume for the search term may be provided. Other momentum analysis data may be displayed as well.

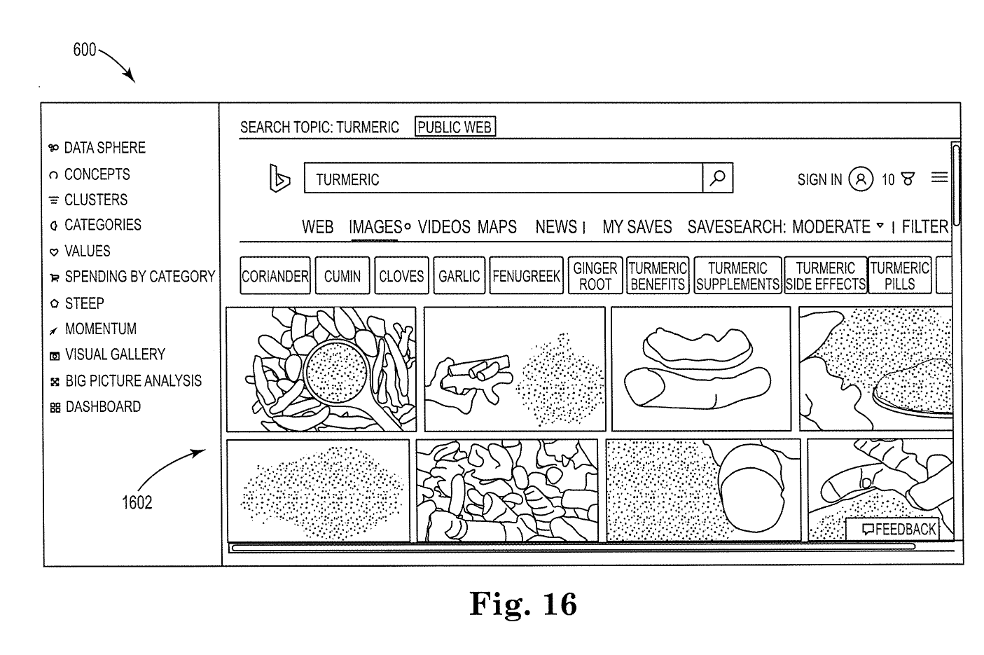

FIG. 16 shows one example of an accessible visual display 1602 of the analysis module, which may provide a visual display of competitive analysis between the search results and other similar brands; a visual display of examples of products or services related to the search topic; a visual display of examples of marketing or packaging related to the search topic; or a visual display of images from the relevant search results. In some embodiments, the visual display 1602 may provide, for example, image search results or images associated with the search term. These images may in some embodiments be displayed chronologically, or by any other suitable organizational manner. In some embodiments, image search results may be analyzed such that the images may be associated with values, categories, core trends, concepts, and/or clusters. For example, source data for the images may be analyzed and scored with respect to values, categories, and/or core trends.

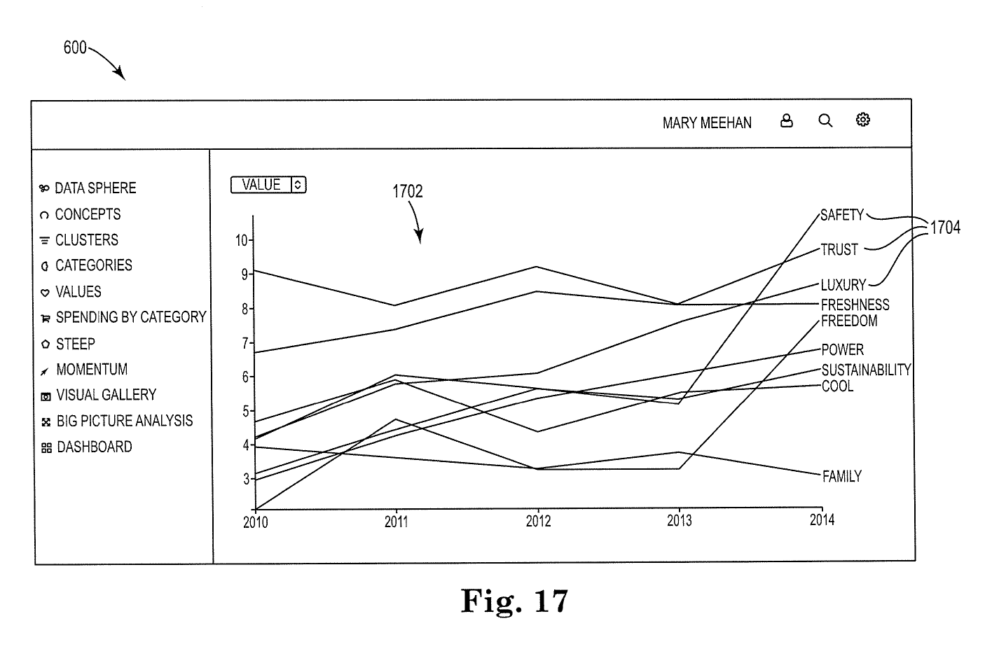

FIG. 17 shows one embodiment of an overall big picture analysis 1702 from the search results displayed by the analysis module within the user interface. In at least one embodiment, the overall analysis 1702 can display how values 1704 have changed in importance or meaning over time to provide a cultural value analysis across multiple values. In at least one embodiment, these selected values 1704 may be based on the search results for the search topic or by user input. The custom collection database may comprise a cultural database that includes the most widely read, e-mailed, or otherwise shared stories from news sources and blogs. These articles may be analyzed with respect to their values using the mapping engine from the system to create cultural value trend maps. In another embodiment, the overall analysis 1702 may compare the core trend maps discussed above with respect to FIG. 14 to these cultural value trend maps. This analysis shows the user how changes in core trends are related to changes in cultural values, which impacts consumer purchasing decisions, voting, and other human behavior. In at least one embodiment, the big picture analysis 1702 can include a comparison between a core trends map and at least one category map and/or at least one value map. The core trends maps and cultural value trend maps can further be compared with the category maps with respect to FIG. 11. These results may be shown as a line graph over time, as seen for example in FIG. 17. However other visual configurations and displays of this data are contemplated by this disclosure. In at least one embodiment, the values can be ranked relative to the core trends. In at least one embodiment, the display may show the generally highest ranked values for the generally highest ranked results for each core trend. In some embodiments, the big picture analysis 1702 may be a static page, while in other embodiments, it may provide real time updates.

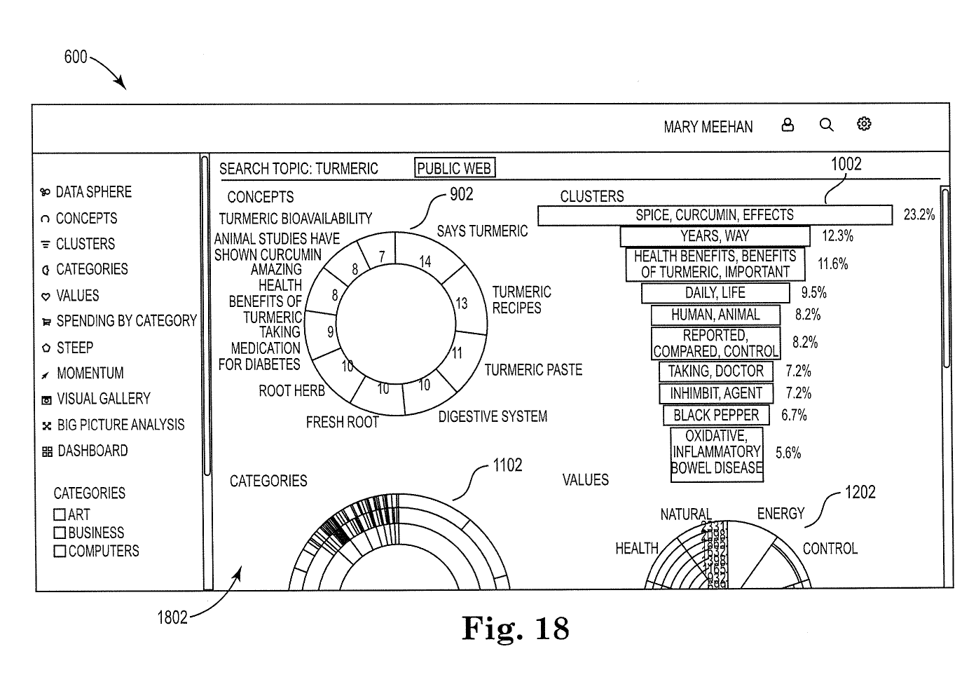

FIG. 18 shows a dashboard view 1802 of the user interface, according to one embodiment. In some embodiments, the dashboard view 1802 may show each of the concept wheel 902, cluster display 1002, categories display 1102, and values display 1202 together on a same screen. In some embodiments, the dashboard view 1802 may provide each of the above-described analysis output displays, including any or all of a data sphere, a concept wheel, a cluster display, a categories display, a values display, a spending display, a core trends analysis, a momentum analysis, a visual gallery, and a big picture analysis. In some embodiments, the dashboard 1802 may be configured to provide a real-time display. For example, the search may be re-run on the one or more databases at intervals, intermittently, or randomly, and the search results may be updated if new or different results are returned. The analyses described above may be performed with respect to the new or different search results, and the dashboard data may be updated to show the new information.

In some embodiments, systems and methods described herein may comprise a tracking feature that allows the user to set up an alert for additional information relating to the documents or data entries. A user may select a specific search topic for tracking and the system may continually, periodically, or at other suitable interval, retrieve new search results for the search function and store the search results into a tracking database. The mapping engine and analysis module may update periodically, or at another suitable interval, to reflect the new search results. In at least one embodiment the analytics module may compare the analysis with the new search results. If the analysis changes, the system may send the user an alert or otherwise flag the changed analysis. In at least one embodiment the analysis module may compare a score for the search term with a new score for the search term. If the score changes, the system may send the user an alert or otherwise flag the changed score. As a particular example, if a top scoring or most relevant value for a search term changes over time, as evidenced by the tracking, an alert may be sent. When the user enters the system, the user may view, through the user interface, displayed information regarding the changes and retrieve the supporting data, which the user may access.

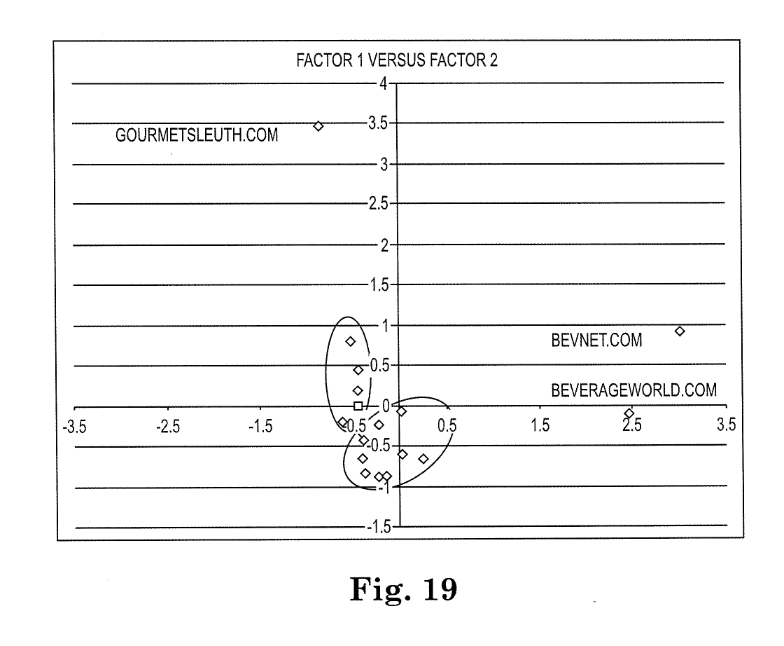

FIG. 19 shows another example output for presenting the search results to a user. Factor analysis may allow a user to see the variability amongst observed values, concepts, and/or core trends. As described above, a factor analysis or correlation analysis may apply one or more statistical algorithms to the determined concepts, categories, values, core trends, and/or signatures to find alignment among them. FIG. 19 shows one example of a factor analysis, wherein scores for two values are plotted together for a plurality of documents. That is, for example, a document's score for a first value is plotted on an X-axis and the same document's score for a second value is plotted on a Y-axis. In some embodiments, a user may have the option to select which values, categories, concepts, and/or core trends to plot together. In other embodiments, most relevant or highest scoring values, categories, concepts, and/or core trends may be plotted together. This may help to show a user how closely the two values, for example, correlate with one another. For example, if a plurality of documents score highly for a pair of values, those values may be closely aligned together.

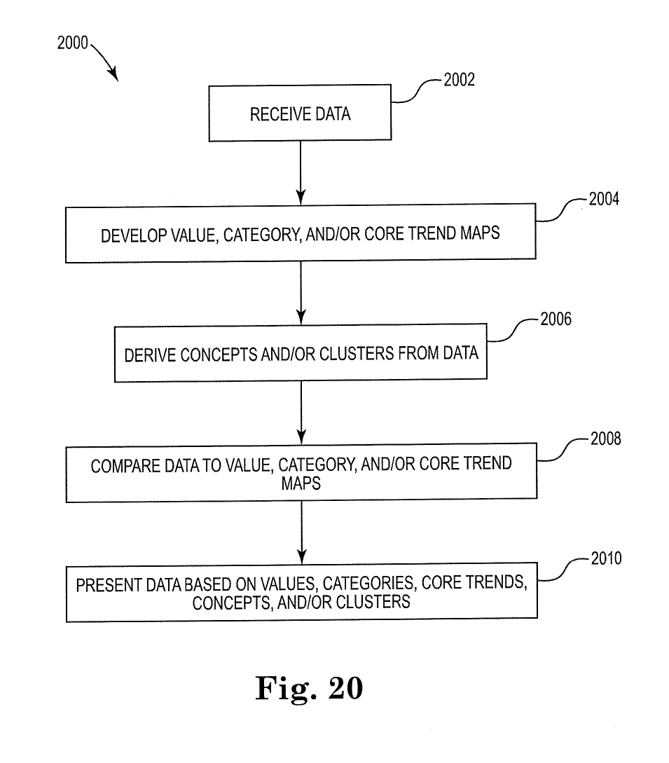

FIG. 20 shows another method 2000, performable by systems of the present disclosure, for providing real time or updated data analysis of product reviews, social media reviews or posts, customer complaints, or other public or private data. The method 2000 may include receiving data 2002; developing value, category, and/or core trend maps 2004; deriving concepts and/or clusters from the data 2006; comparing the data to the maps 2008; and presenting the data with meaning based on values, categories, core trends, concepts, and/or clusters 2010. In other embodiments, the method 2000 may include additional and/or alternative steps. Moreover, these steps may be performed in any suitable order, and are not limited to the order shown in FIG. 20.

As indicated above, the method 2000 may include receiving data 2002. The data may be private or public company data in some embodiments. Moreover, the data may include a plurality of data entries or records. For example, the data may be provided as a database of reviews, complaints, social media posts, or other data related to a company or a company's product. The data may be provided by the company to which it pertains, or it may be publicly accessible or retrievable. In some embodiments, the data may be received from multiple databases, such as multiple social media databases, for example. In some embodiments, the data may consist of historical data, such as historical reviews, complaints, social media posts, or other data related to a company or a company's product.

The method 2000 may additionally include developing value, category, and/or core trend maps 2004. As described above, values, categories, and/or core trends may be words or phrases that may convey an idea, feeling, or other parameter. The maps may be developed based on documents in a custom collections database, for example, which may be selected based on their relevance to a particular value, category, or core trend. In some embodiments, a map may include a series of words, phrases, or semantic combinations that correlate with the particular value, category, or core trend. In some embodiments, a map may be developed for each value, category, and core trend. The values, categories, and or core trends for which maps are created may be predefined in some embodiments. The values, categories, and/or core trends may be defined by one or more standards, or may be defined by a company, for example. In one particular example, a company may wish to organize customer complaints by responding department, and thus may define categories as “information technology,” “billing,” and/or other departments. By evaluating the complaints received in the received data, maps may be developed for each of these custom categories. In some embodiments, the maps may be created based on a portion of the received data. For example, where the received data contains 1000+ customer complaints, the data maps may be developed based on an analysis of 100, or another suitable number of, customer complaints.

The method 2000 may additionally include deriving concepts and/or clusters from the received data 2006. As described above, for example, a clustering tool such as Carrot2, a clustering engine that uses the Lingo Clustering Algorithm, LingPipe, another open source clustering engine, or any other suitable clustering tool may be used to derive concepts and clusters from the received data. In some embodiments, the data may be organized or grouped by clusters and/or concepts.

In some embodiments, presenting the data may include comparing each of the data entries or records in the received data to the developed value or category maps 2008 to determine correlation between the data and the values, categories, and core trends. As indicated above, a mapping engine, for example, may compare each data entries or documents to each value, category, and core trend map. Semantics tools may be used to compare the documents or data entries to the maps to determine a correlation between each data and each map.

In some embodiments, similar to step 510 described above with respect to FIG. 5, comparing the data to the value, category, and/or core trend maps may include assigning a score to each data entry or document with respect to each value, category, and/or core trend. For example, each document may be assigned a score with respect to the value of energy. The score may be determined by comparing each document with the map for the value of energy. Algorithms such as, but not limited to, Naïve Bayes may be used to compare the document with the map to produce a numbered score, for example. The score may signal how strongly the document correlates with the value of energy. In some embodiments, a higher score may signal a stronger correlation with the value, category, or core trend, whereas a lower score may signal a weaker correlation with the value, category, or core trend. Each data entry or document may thus be assigned multiple scores, one for each value, category, and/or core trend. As indicated above, in some embodiments, a threshold may be applied to the value, category, and/or core trend scores. A threshold may determine whether each document or data entry will be presented as associated with a particular value, category, or core trend. If a document meets or exceeds the threshold for the value of energy, for example, the document may be associated with the value of energy. In some embodiments, the scores assigned to a document or data entry may be ordered, such that the value, category, and/or core trend having the highest correlation to the document may be selected as a predominant or most relevant value, category, or core trend for that document or data entry.

In at least one embodiment, each relevant document or data entry may be limited to matching with a maximum number of values, categories, and/or core trends, which may be a pre-selected or pre-configured number, and may be configurable by a user. For example, in one embodiment, each document or data entry may be matched with a maximum of five categories and five values.

The method 2000 may additionally include presenting the received data in terms of values, categories, concepts, and/or clusters 2010. The data may generally be presented according to the values, categories, clusters, and/or concepts. In some embodiments, the data may be presented via a dynamic, interactive user interface, such as via a dashboard view. In some embodiments, any or all of the outputs described above with respect to FIGS. 8-18 may be used to present the data with meaning according to values, categories, concepts, and/or clusters. In other embodiments, the data may be presented via a report or other output. The data may also be presented in terms of trends and other organizational tools.

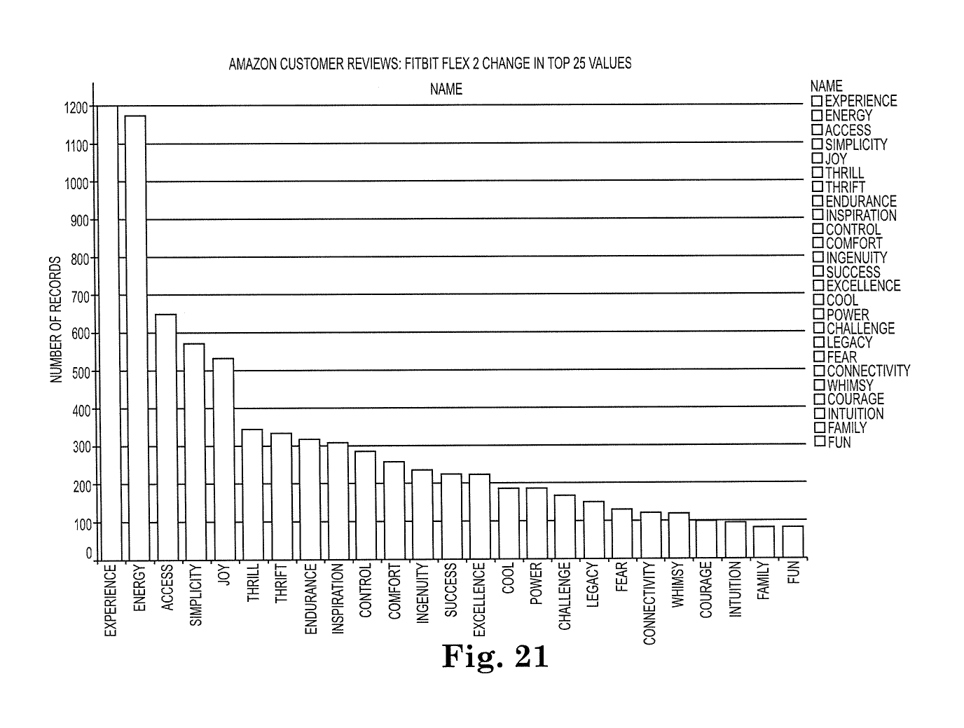

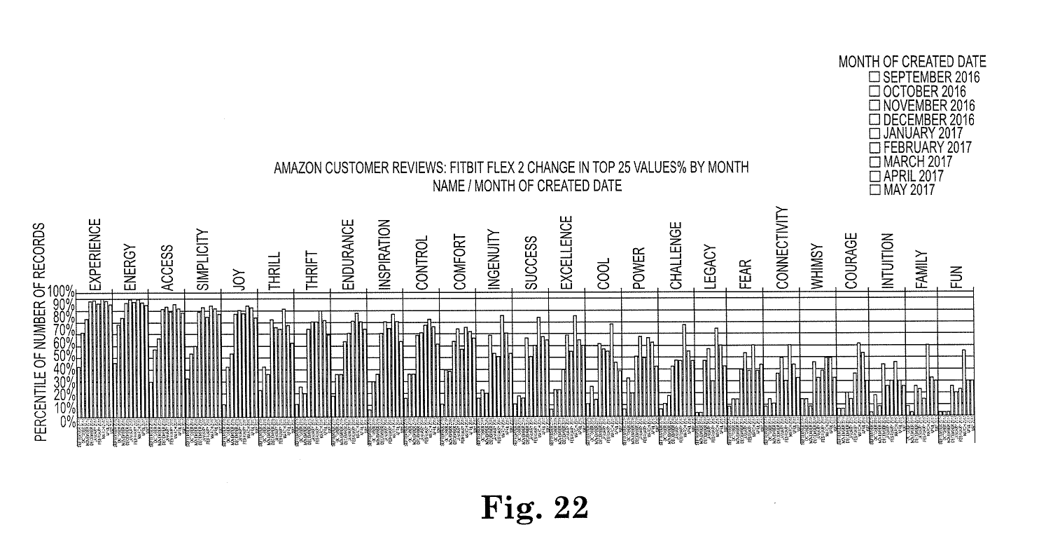

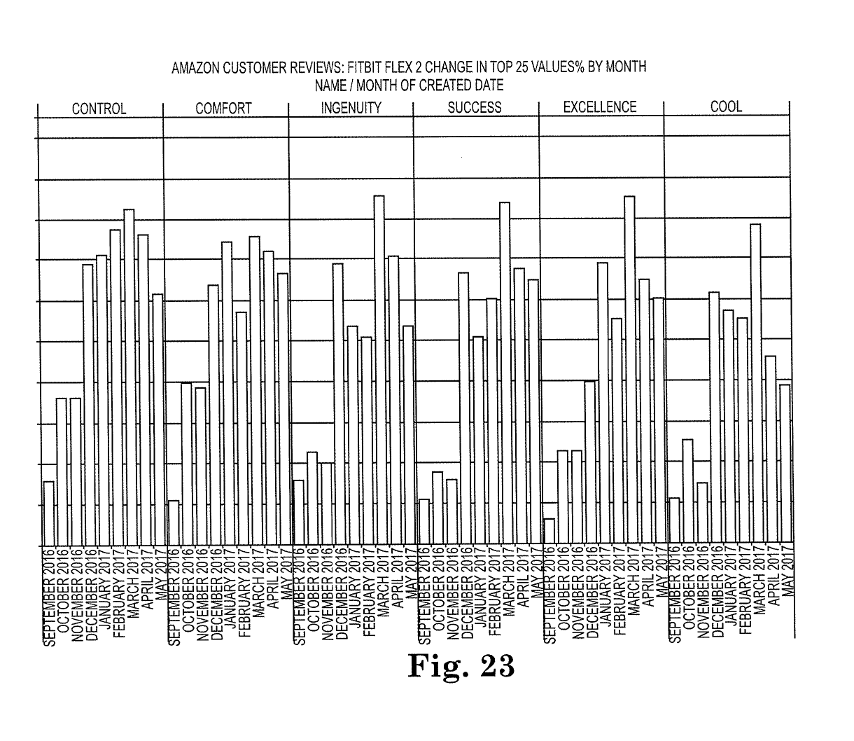

FIGS. 21-23 demonstrate a real-world example of the method according to one or more embodiments, performed using real-world data. In this particular example, publicly available customer reviews on Amazon for a product, the Fitbit Flex 2, were analyzed and tracked over time. The reviews were initially analyzed and mapped to several values, including experience, energy, access, simplicity, joy, thrill, thrift, endurance, inspiration, control, comfort, ingenuity, success, excellence, cool, power, challenge, legacy, fear, connectivity, whimsy, courage, intuition, family, fun, and other values. FIG. 21 demonstrates the distribution of the 25 most prevalent values among the Amazon reviews. This type of distribution display may allow a company, such as Fitbit, to see how their customers are generally experiencing and responding to their product. Over time, the Amazon reviews for the Fitbit Flex 2 may be monitored or tracked, such that the analysis may be rerun or updated with respect to the same values. Over time, as the most prevalent and least prevalent values change based on newly received reviews, trends in the reviews may be tracked. FIG. 22 shows an example of a bar graph, where each of the 25 most prevalent values is measured over a period of nine months. For each month, the percent of reviews correlating with each value is shown. FIG. 23 shows a closer view of the chart of FIG. 22, where some of the values may be seen more closely.

Systems and methods of the present disclosure may generally allow a user to view and understand data in a more comprehensive manner. For example, the user may have the ability to view common themes or trends among data. The analyses described herein may additionally allow a user to view changes to data over time. By comparing values, categories, clusters, concepts, and/or other data, such as BLS spending data, a user may have the ability to see new market opportunities, opportunities for better customer relationships or understanding, and other information. In general, the systems and methods of the present disclosure may allow a user to develop a comprehensive understanding a large quantity of data or data entries in an efficient and effective manner, without the need to read, review, or analyze individual data entries manually.

For purposes of this disclosure, any system described herein may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, a system or any portion thereof may be a minicomputer, mainframe computer, personal computer (e.g., desktop or laptop), tablet computer, mobile device (e.g., personal digital assistant (PDA) or smart phone) or other hand-held computing device, server (e.g., blade server or rack server), a network storage device, or any other suitable device or combination of devices and may vary in size, shape, performance, functionality, and price. A system may include volatile memory (e.g., random access memory (RAM)), one or more processing resources such as a central processing unit (CPU) or hardware or software control logic, ROM, and/or other types of nonvolatile memory (e.g., EPROM, EEPROM, etc.). A basic input/output system (BIOS) can be stored in the non-volatile memory (e.g., ROM), and may include basic routines facilitating communication of data and signals between components within the system. The volatile memory may additionally include a high-speed RAM, such as static RAM for caching data.

Additional components of a system may include one or more disk drives or one or more mass storage devices, one or more network ports for communicating with external devices as well as various input and output (I/O) devices, such as a keyboard, a mouse, touchscreen and/or a video display. Mass storage devices may include, but are not limited to, a hard disk drive, floppy disk drive, CD-ROM drive, smart drive, flash drive, or other types of non-volatile data storage, a plurality of storage devices, a storage subsystem, or any combination of storage devices. A storage interface may be provided for interfacing with mass storage devices, for example, a storage subsystem. The storage interface may include any suitable interface technology, such as EIDE, ATA, SATA, and IEEE 1394. A system may include what is referred to as a user interface for interacting with the system, which may generally include a display, mouse or other cursor control device, keyboard, button, touchpad, touch screen, stylus, remote control (such as an infrared remote control), microphone, camera, video recorder, gesture systems (e.g., eye movement, head movement, etc.), speaker, LED, light, joystick, game pad, switch, buzzer, bell, and/or other user input/output device for communicating with one or more users or for entering information into the system. These and other devices for interacting with the system may be connected to the system through I/O device interface(s) via a system bus, but can be connected by other interfaces such as a parallel port, IEEE 1394 serial port, a game port, a USB port, an IR interface, etc. Output devices may include any type of device for presenting information to a user, including but not limited to, a computer monitor, flat-screen display, or other visual display, a printer, and/or speakers or any other device for providing information in audio form, such as a telephone, a plurality of output devices, or any combination of output devices.

A system may also include one or more buses operable to transmit communications between the various hardware components. A system bus may be any of several types of bus structure that can further interconnect, for example, to a memory bus (with or without a memory controller) and/or a peripheral bus (e.g., PCI, PCIe, AGP, LPC, etc.) using any of a variety of commercially available bus architectures.

One or more programs or applications, such as a web browser and/or other executable applications, may be stored in one or more of the system data storage devices. Generally, programs may include routines, methods, data structures, other software components, etc., that perform particular tasks or implement particular abstract data types. Programs or applications may be loaded in part or in whole into a main memory or processor during execution by the processor. One or more processors may execute applications or programs to run systems or methods of the present disclosure, or portions thereof, stored as executable programs or program code in the memory, or received from the Internet or other network. Any commercial or freeware web browser or other application capable of retrieving content from a network and displaying pages or screens may be used. In some embodiments, a customized application may be used to access, display, and update information. A user may interact with the system, programs, and data stored thereon or accessible thereto using any one or more of the input and output devices described above.

A system of the present disclosure can operate in a networked environment using logical connections via a wired and/or wireless communications subsystem to one or more networks and/or other computers. Other computers can include, but are not limited to, workstations, servers, routers, personal computers, microprocessor-based entertainment appliances, peer devices, or other common network nodes, and may generally include many or all of the elements described above. Logical connections may include wired and/or wireless connectivity to a local area network (LAN), a wide area network (WAN), hotspot, a global communications network, such as the Internet, and so on. The system may be operable to communicate with wired and/or wireless devices or other processing entities using, for example, radio technologies, such as the IEEE 802.xx family of standards, and includes at least Wi-Fi (wireless fidelity), WiMax, and Bluetooth wireless technologies. Communications can be made via a predefined structure as with a conventional network or via an ad hoc communication between at least two devices.

Hardware and software components of the present disclosure, as discussed herein, may be integral portions of a single computer or server or may be connected parts of a computer network. The hardware and software components may be located within a single location or, in other embodiments, portions of the hardware and software components may be divided among a plurality of locations and connected directly or through a global computer information network, such as the Internet. Accordingly, aspects of the various embodiments of the present disclosure can be practiced in distributed computing environments where certain tasks are performed by remote processing devices that are linked through a communications network. In such a distributed computing environment, program modules may be located in local and/or remote storage and/or memory systems.

As will be appreciated by one of skill in the art, the various embodiments of the present disclosure may be embodied as a method (including, for example, a computer-implemented process, a business process, and/or any other process), apparatus (including, for example, a system, machine, device, computer program product, and/or the like), or a combination of the foregoing. Accordingly, embodiments of the present disclosure may take the form of an entirely hardware embodiment, an entirely software embodiment (including firmware, middleware, microcode, hardware description languages, etc.), or an embodiment combining software and hardware aspects. Furthermore, embodiments of the present disclosure may take the form of a computer program product on a computer-readable medium or computer-readable storage medium, having computer-executable program code embodied in the medium, that define processes or methods described herein. A processor or processors may perform the necessary tasks defined by the computer-executable program code. Computer-executable program code for carrying out operations of embodiments of the present disclosure may be written in an object oriented, scripted or unscripted programming language such as Java, Perl, PHP, Visual Basic, Smalltalk, C++, or the like. However, the computer program code for carrying out operations of embodiments of the present disclosure may also be written in conventional procedural programming languages, such as the C programming language or similar programming languages. A code segment may represent a procedure, a function, a subprogram, a program, a routine, a subroutine, a module, an object, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

In the context of this document, a computer readable medium may be any medium that can contain, store, communicate, or transport the program for use by or in connection with the systems disclosed herein. The computer-executable program code may be transmitted using any appropriate medium, including but not limited to the Internet, optical fiber cable, radio frequency (RF) signals or other wireless signals, or other mediums. The computer readable medium may be, for example but is not limited to, an electronic, magnetic, optical, electromagnetic, infrared, or semiconductor system, apparatus, or device. More specific examples of suitable computer readable medium include, but are not limited to, an electrical connection having one or more wires or a tangible storage medium such as a portable computer diskette, a hard disk, a random access memory (RAM), a read-only memory (ROM), an erasable programmable read-only memory (EPROM or Flash memory), a compact disc read-only memory (CD-ROM), or other optical or magnetic storage device. Computer-readable media includes, but is not to be confused with, computer-readable storage medium, which is intended to cover all physical, non-transitory, or similar embodiments of computer-readable media.

Various embodiments of the present disclosure may be described herein with reference to flowchart illustrations and/or block diagrams of methods, apparatus (systems), and computer program products. It is understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular special purpose machine, such that the code portions, which execute via the processor of the computer or other programmable data processing apparatus, create mechanisms for implementing the functions/acts specified in the flowchart and/or block diagram block or blocks. Alternatively, computer program implemented steps or acts may be combined with operator or human implemented steps or acts in order to carry out an embodiment of the invention.

Additionally, although a flowchart or block diagram may illustrate a method as comprising sequential steps or a process as having a particular order of operations, many of the steps or operations in the flowchart(s) or block diagram(s) illustrated herein can be performed in parallel or concurrently, and the flowchart(s) or block diagram(s) should be read in the context of the various embodiments of the present disclosure. In addition, the order of the method steps or process operations illustrated in a flowchart or block diagram may be rearranged for some embodiments. Similarly, a method or process illustrated in a flow chart or block diagram could have additional steps or operations not included therein or fewer steps or operations than those shown. Moreover, a method step may correspond to a method, a function, a procedure, a subroutine, a subprogram, etc.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

We claim:

1. A system for analyzing and presenting data, the system comprising:
a mapping engine in communication with an information database comprising a plurality of records, wherein the mapping engine:
creates a map defining a value, category, or core trend from at least a first record by applying a text mining tool to the at least a first record;
determines whether a second record relates to the value, category, or core trend by comparing the second record to the map using a semantics analysis;
based on the semantics analysis, assigns a score to the second record, the score signaling how well the second record relates to the value, category, or core trend; and
based on the score, associates the second record with the value, category, or core trend; and
an analysis module in data communication with the mapping engine, wherein the analysis module creates graphic visualizations of records associated with the value, category, or core trend, and presents the graphic visualizations via a user interface.

2. The system of claim 1, wherein associating the second record with the value, category, or core trend comprises comparing the score to a threshold, and, if the score meets or exceeds the threshold, associating the second record with the value, category, or core trend.

3. The system of claim 1, further comprising a custom collections database comprising a plurality of records from which the mapping engine creates the map.

4. The system of claim 3, wherein the custom collections database comprises a values library, a categories library, and a core trends library.

5. The system of claim 4, wherein the mapping engine further stores the map in the custom collections database.

6. The system of claim 1, further comprising a clustering engine in data communication with the information database, wherein the clustering engine derives a concept and/or a cluster from the second document.

7. The system of claim 1, wherein the mapping engine creates a map for each of a plurality of values, a plurality of categories, and a plurality of core trends.

8. The system of claim 1, wherein the user interface is configured to receive a search term entered by a user for searching the information database.

9. The system of claim 8, wherein the second record comprises a search result from a search of the information database.

10. A method of retrieving and interpreting search results from a search of an information database storing a plurality of records, the method comprising:
creating a map by applying a text mining tool to a plurality of records from the information database, the map defining a value, category, or core trend;
receiving a search term at a user interface;
searching the information database for the search term to generate a plurality of search results;
determining whether the search results relate to the value, category, or core trend by comparing each of the search results to the map using a semantics analysis;
based on the semantics analysis, assigning a score to each search result, the score signaling how well the search result relates to the value, category, or core trend;
based on the assigned scores, associating at least one of the search results with the value, category, or core trend; and
presenting the search results, at the user interface, based on associated concepts, clusters, values, categories, and/or core trends.

11. The method of claim 10, further comprising deriving concepts and/or clusters from the search results using a clustering engine.

12. The method of claim 10, further comprising storing the map in a custom collections database comprising the plurality of records from which the mapping engine creates the map.

13. The method of claim 12, wherein the custom collections database comprises a values library, a categories library, and a core trends library.

14. The method of claim 10, further comprising creating a map for each of a plurality of values, a plurality of categories, and a plurality of core trends.

15. A method of analyzing a plurality of data records, the method comprising;
receiving the plurality of data records;
creating a map by applying a text mining tool to a plurality of the data records, the map defining a value, category, or core trend;
determining whether the data records relate to the value, category, or core trend by comparing each data record to the map using a semantics analysis;
based on the semantics analysis, assigning a score to each data record, the score signaling how well the data record relates to the value, category, or core trend;
based on the assigned scores, associating at least one of the data records with the value, category, or core trend; and
presenting the data records, at a user interface, based on associated concepts, clusters, values, categories, and/or core trends.

16. The method of claim 15, further comprising deriving concepts and/or clusters from the data records using a clustering engine.

17. The method of claim 15, further comprising storing the map in a custom collections database comprising the plurality of data records from which the mapping engine creates the map.

18. The method of claim 15, further comprising creating a map for each of a plurality of values, a plurality of categories, and a plurality of core trends.
